(19) United States
(12) Patent Application Publication
(10) Pub. No.: US 2025/0339040 A1
(43) Pub. Date: Nov. 6, 2025

(54) DEVICE AND METHOD FOR MONITORING LEFT ATRIAL PRESSURE

(71) Applicant: EDWARDS LIFESCIENCES CORPORATION, Irvine, CA (US)

(72) Inventors: Hao Shang, Irvine, CA (US); Zheng Lin, Fountain Valley, CA (US)

(21) Appl. No.: 19/272,903

(22) Filed: Jul. 17, 2025

Related U.S. Application Data
(63) Continuation of application No. PCT/US2024/012377, filed on Jan. 22, 2024.
(60) Provisional application No. 63/481,127, filed on Jan. 23, 2023.

(51) Int. Cl. A61B 5/0215; A61B 5/00; A61B 5/07

(52) U.S. Cl. CPC A61B 5/02152; A61B 5/0004; A61B 5/02158; A61B 5/076; A61B 5/686

(57) ABSTRACT

A cardiac device configured to be positioned in a heart includes a body configured to be positioned in a wall between a right pulmonary vein and a right atrium or a superior vena cava and a sensor attached to the cardiac device. The sensor includes a sensor housing, a transducer, and control circuitry in the sensor housing in electrical communication with the transducer that is configured to sense a signal from a fluid in the right pulmonary vein, the right atrium, or the superior vena cava or a signal from the heart.

### CROSS-REFERENCE TO RELATED APPLICATION(S)

This application is a continuation of International Application No. PCT/US2024/012377, filed Jan. 22, 2024, which claims the benefit of U.S. Provisional Application No. 63/481,127, filed Jan. 23, 2023, the disclosures of which are hereby incorporated by reference in their entireties.

### BACKGROUND

The present disclosure relates to implantable medical devices, and in particular, to implantable cardiac sensors.

Implantable cardiac sensors can be important to proper diagnosis and treatment of many heart diseases. Implantation of these cardiac sensors enables health data to be collected over a longer period compared to non-implantable sensors, thereby permitting physicians to make more informed patient care decisions. Among these cardiac sensors are cardiac pressure sensors which can measure the pressure of the heart's chambers. Information from these cardiac pressure sensors can be valuable for detection and diagnosis of heart diseases. Implantation of these sensors can be difficult and/or preclude certain future treatments. Therefore, solutions regarding implantation which reduce the complexity of implantation and do not preclude certain future treatments is desired.

### SUMMARY

A cardiac device includes a body configured to be positioned in a wall between a right pulmonary vein and a right atrium or a superior vena cava and a sensor attached to the cardiac device. The sensor includes a sensor housing, a transducer, and control circuitry in the sensor housing in electrical communication with the transducer that is configured to sense a signal from a fluid in the right pulmonary vein, the right atrium, or the superior vena cava or a signal from a heart.

A cardiac device includes a body configured to be positioned in a wall between a vein fluidly coupled to a left atrium and a right atrium or a vein fluidly coupled to the right atrium, and a sensor attached to the cardiac device. The sensor includes a sensor housing, a transducer, and control circuitry in the sensor housing in electrical communication with the transducer that is configured to sense a signal from a fluid in the vein fluidly coupled to the left atrium, the right atrium, or the vein fluidly coupled to the right atrium or a signal from a heart.

A method of determining a cardiac pressure includes inserting a catheter to a left atrium of a heart, and advancing the catheter into a right pulmonary vein to a wall between the right pulmonary vein and a superior vena cava or a right atrium. An opening is formed in the wall between the right pulmonary vein and the superior vena cava or the right atrium, and a cardiac device with a sensor is deployed into the opening. A pressure is sensed in the right pulmonary vein.

### BRIEF DESCRIPTION OF THE DRAWINGS

FIG. 1 is an anterior view of an example heart.

FIG. 2 is a posterior view of the example heart.

FIG. 3 is a frontal cross-sectional view of the example heart.

FIG. 4 is a superior cross-sectional view of the example heart, taken along line 4-4 of FIG. 3.

FIG. 5A is a frontal cross-sectional view of the example heart having a pulmonary vein opening.

FIG. 5B is a superior cross-sectional view of the example heart, taken along line 5B-5B of FIG. 5A, having the pulmonary vein opening.

FIG. 6A is a block diagram of a first example of a sensor device.

FIG. 6B is a block diagram of a second example of a sensor device.

FIG. 7 is a superior cross-sectional view of the heart having a first example of a cardiac device with a sensor implanted therein.

FIG. 8A is a superior cross-sectional view of the heart having a second example of a cardiac device with a sensor on a first end implanted therein.

FIG. 8B is a superior cross-sectional view of the heart having the second example of the cardiac device with a sensor on a second end implanted therein.

FIG. 8C is a superior cross-sectional view of the heart having the second example of the cardiac device with a sensor extending through the cardiac device implanted therein.

FIG. 9 is a superior cross-sectional view of the heart having a third example of a cardiac device with a sensor extending the cardiac device implanted therein.

FIG. 10 is a side view of a stent-type cardiac device with a sensor extending through the cardiac device.

FIG. 11 is a superior cross-sectional view of the heart having a fourth example of a cardiac device.

FIG. 12 is a perspective view of a grommet-type cardiac device with a sensor extending through the cardiac device.

FIG. 13 is a flow diagram illustrating an example method for forming a pulmonary vein opening.

FIG. 14 is a cutaway view of the heart and associated vasculature showing example catheter access paths for pulmonary vein procedures.

FIGS. 15A-15D are cross-sectional views of the heart showing a catheter advancing through the heart.

FIGS. 16A and 16B are posterior views of the heart having cardiac devices in right inferior pulmonary veins.

FIG. 17 is a posterior view of the heart having a cardiac device in a right superior pulmonary vein and a right pulmonary artery.

FIG. 18 is a posterior view of the heart having a sensor in a left superior pulmonary vein and a left pulmonary artery in accordance with one or more examples.

### DETAILED DESCRIPTION

The present disclosure relates to devices, systems, and methods for implanting a cardiac device between a pulmonary vein and a right-side chamber or vessel of a heart. The cardiac device includes a sensor that is capable of measuring characteristics of the fluid, for example blood, contacting the cardiac device and/or sensor. The characteristics can include pressure, flow, temperature, oxygen saturation, glucose, or any other characteristic that one of skill in the art could measure from blood. Aspects of the present disclosure relate to the anatomy of the heart, thus a description of relevant cardiac anatomy is presented herein.

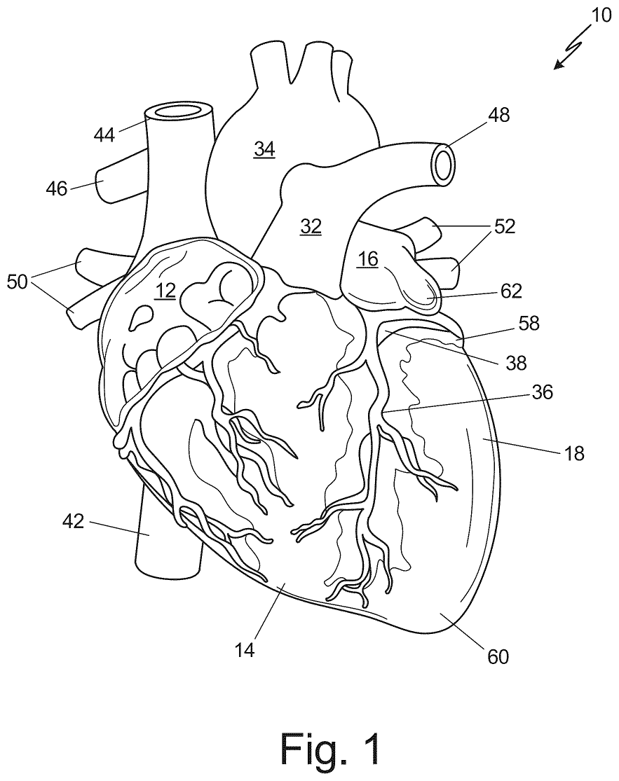

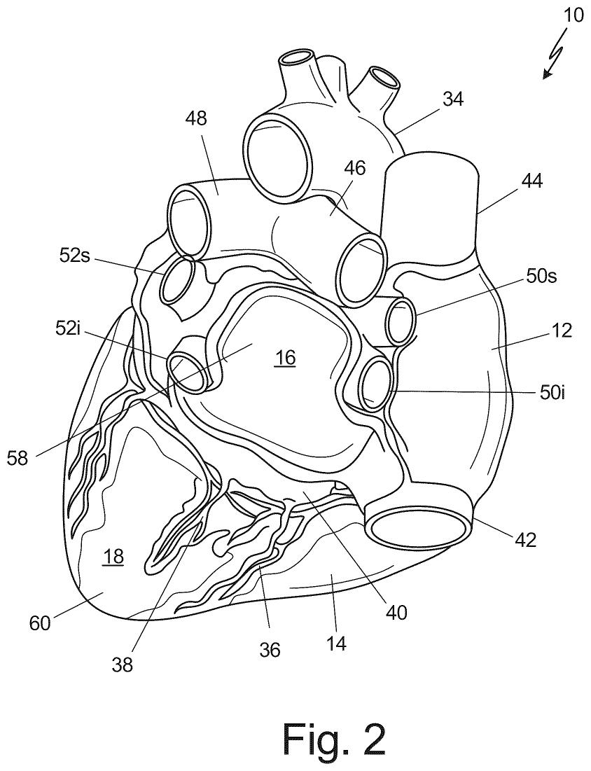

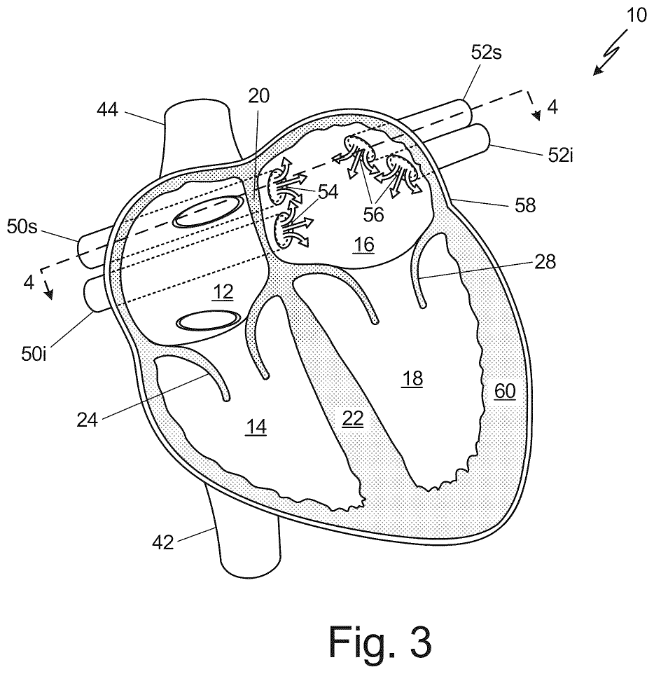

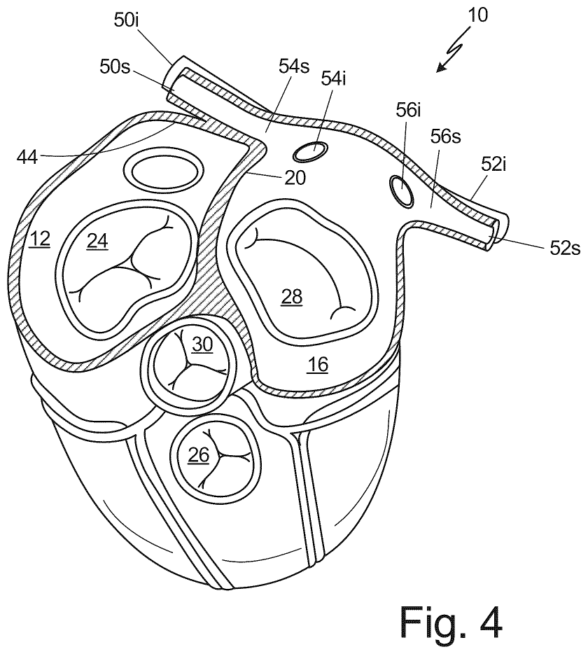

FIG. 1 is an anterior view of heart 10. FIG. 2 is a posterior view of heart 10. FIG. 3 is a frontal cross-sectional view of heart 10. FIG. 4 is a superior cross-sectional view of heart 10, taken along line 4-4 of FIG. 3. FIGS. 1-4 will be discussed together. Heart 10 can be any mammalian heart, specifically including a human heart. FIGS. 1-4 show heart 10, right atrium 12, right ventricle 14, left atrium 16, left ventricle 18, atrial septum 20, ventricle septum 22, tricuspid valve 24, pulmonary valve 26, mitral valve 28, aortic valve 30, pulmonary artery 32, aorta 34, coronary arteries 36, coronary veins 38, coronary sinus 40, inferior vena cava 42, superior vena cava 44, right pulmonary arteries 46, left pulmonary arteries 48, right pulmonary veins 50 (including right inferior pulmonary vein 50i and right superior pulmonary vein 50s), left pulmonary veins 52 (including left inferior pulmonary vein 52i and left superior pulmonary vein 52s), right pulmonary vein ostia 54 (including right inferior pulmonary vein ostium 54i and right superior pulmonary vein ostium 54s), left pulmonary vein ostia 56 (including left inferior pulmonary vein ostium 56i and left superior pulmonary vein ostium 56s), base 58, apex 60, and left atrial appendage 62.

With reference to FIGS. 1-4, heart 10 includes four chambers, namely right atrium 12, right ventricle 14, left atrium 16, and left ventricle 18. A wall of muscle, referred to as the septum, separates the right-side chambers from the left-side chambers. In particular, atrial septum 20 separates right atrium 12 from left atrium 16, and ventricle septum 22 separates right ventricle 14 from left ventricle 18.

Heart 10 further includes four valves for aiding the circulation of blood therein. Heart valves generally comprise a relatively dense fibrous ring, referred to herein as the annulus, as well as a plurality of leaflets or cusps attached to the annulus. Generally, the size and position of the leaflets or cusps can be such that when the heart contracts, the resulting increased blood pressure produced within the corresponding heart chamber forces the leaflets at least partially open to permit flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage.

Heart 10 include tricuspid valve 24, pulmonary valve 26, mitral valve 28, and aortic valve 30. Tricuspid valve 24 separates right atrium 12 from right ventricle 14. Tricuspid valve 24 generally has three cusps or leaflets and generally closes during ventricular contraction (i.e., systole) and open during ventricular expansion (i.e., diastole). Pulmonary valve 26 separates right ventricle 14 from pulmonary artery 32 and can be configured to open during systole so that blood can be pumped toward the lungs and close during diastole to prevent blood from leaking back into heart 10 from pulmonary artery 32. Pulmonary valve 26 generally has three cusps/leaflets, wherein each one can have a crescent-type shape. Mitral valve 28 generally has two cusps/leaflets and separates left atrium 16 from left ventricle 18. Mitral valve 28 can generally be configured to open during diastole so that blood in left atrium 16 can flow into left ventricle 18 and closes during diastole to prevent blood from leaking back into left atrium 16. Aortic valve 30 separates left ventricle 18 from aorta 34. Aortic valve 30 is configured to open during systole to permit blood leaving left ventricle 18 to enter aorta 34 and close during diastole to prevent blood from leaking back into left ventricle 18. Aortic valve 30 generally has three cusps/leaflets.

Tricuspid valve 24 and mitral valve 28, together known as the atrioventricular valves, are generally associated with a sub-valvular apparatus (not shown), including a collection of chordae tendineae and papillary muscles securing the leaflets of the respective valves to promote and/or facilitate proper coaptation of the valve leaflets and prevent prolapse thereof. The papillary muscles generally comprise finger-like projections from the ventricle walls. The chordac tendineac generally keep the valve leaflets from opening in the wrong direction, thereby preventing blood to flow backwards.

Surrounding right ventricle 14 and left ventricle 18 are a number of coronary arteries 36 that supply oxygenated blood to the heart muscle and a number of coronary veins 38 that return the blood from the heart muscle to right atrium 12 via coronary sinus 40. Coronary sinus 40 is a relatively large vein that extends generally around the upper portion of left ventricle 18 and provides a return conduit for blood returning to right atrium 12.

Deoxygenated blood enters right atrium 12 through inferior vena cava 42 and superior vena cava 44. The right side (i.e., right atrium 12 and right ventricle 14) of heart 10 then pumps this deoxygenated blood into pulmonary artery 32. Pulmonary artery 32 branches off into right pulmonary arteries 46 and left pulmonary arteries 48. Right pulmonary arteries 46 and left pulmonary arteries 48 carry the deoxygenated blood from the right side of the heart to the lungs. Fresh oxygen enters the blood stream in the lungs, and the blood moves to the left side of heart 10 via right pulmonary veins 50 and left pulmonary veins 52 that ultimately terminate at left atrium 16.

The primary roles of the chambers of the left side of heart 10 (i.e., left atrium 16 and left ventricle 18) are to act as holding chambers for blood returning from the lungs (not shown) and to act as a pump to transport blood to the body. Left atrium 16 receives oxygenated blood from the lungs via right pulmonary veins 50 and left pulmonary veins 52. The oxygenated blood that is collected from right pulmonary veins 50 and left pulmonary veins 52 in left atrium 16 enters left ventricle 18 through mitral valve 28. In some patients, the walls of left atrium 16 are slightly thicker than the walls of right atrium 12.

Right pulmonary veins 50 carry blood from the right lung (not shown) to left atrium 16, and left pulmonary veins 52 carry blood from the left lung (not shown) to left atrium 16. The blood is distributed to the rest of the circulatory system from left atrium 16, as described in detail herein. Right pulmonary veins 50 include right inferior pulmonary vein 50i and right superior pulmonary vein 50s, as shown. Left pulmonary veins 52 include left inferior pulmonary vein 52i and left superior pulmonary vein 52s. Right pulmonary vein ostia 54 and left pulmonary vein ostia 56 of right pulmonary veins 50 and left pulmonary veins 52, respectively, are generally located at or near the posterior left atrial wall of left atrium 16. Right pulmonary vein ostia 54 include right inferior pulmonary vein ostium 54i of right inferior pulmonary vein 50i and right superior pulmonary vein ostium 54s of right superior pulmonary vein 50s. Left pulmonary vein ostia 56 include left inferior pulmonary vein ostium 56i of left inferior pulmonary vein 52i and left superior pulmonary vein ostium 56s of left superior pulmonary vein 52s.

Left ventricle 18 is the primary pumping chamber of heart 10. A healthy left ventricle 18 is longer than a width of left ventricle 18. A length can be with respect to the mean electrical axis of the heart and a width can be with respect to a transverse axis extending between opposing walls of left ventricle 18 at the widest point. Heart 10 descends from base 58 with a decreasing cross-sectional diameter and/or circumference to apex 60. Generally, the apical region of heart 10 can be considered the bottom region of heart 10 that is within the left and/or right ventricular region but is distal to mitral valve 28 and tricuspid valve 24 and toward a tip of heart 10.

The pumping of blood from left ventricle 18 is accomplished by a squeezing motion and a twisting or torsional motion. The squeezing motion occurs between the lateral walls of left ventricle 18 and ventricle septum 22. The twisting motion is a result of contraction of heart muscle fibers that extend in a generally circular or spiral direction around heart 10. When these fibers contract, they produce a gradient of angular displacements of the myocardium from apex 60 to base 58 about the mean electrical axis of the heart. The resultant force vectors extend at angles from about 30-60 degrees to the flow of blood through aortic valve 30 and aorta 34. The contraction of heart 10 is manifested as a counterclockwise rotation of apex 60 relative to base 58, when viewed from apex 60 (i.e., inferior view of heart 10). The contractions of heart 10, in connection with the filling volumes of left atrium 16 and left ventricle 18, respectively, can result in relatively high fluid pressures in the left side of heart 10 at least during certain phase(s) of the cardiac cycle. Attached to left atrium 16 is left atrial appendage 62, which generally comprise a muscular car-shaped pouch. Left atrial appendage 62 is thought to function as a decompression chamber during left ventricular systole and during other periods when left atrial pressure is high.

Fluid volume and pressure conditions associated with the various cardiac chambers and anatomy described above can impact the health of a patient. For example, congestive heart failure is a condition associated with the relatively slow movement of blood through the heart and/or body, which can cause the fluid pressure in one or more chambers of the heart to increase, particularly in the left side of the heart. For example, when left ventricle 18 fails or when mitral valve 28 fails, left atrial pressure can increase substantially. As a result, heart 10 may not pump sufficient oxygen to meet the body's needs. Increased left atrial volume and pressure can further result in abnormal P waves in cardiac electrical signals.

The various chambers of heart 10 can respond to pressure increases by stretching to hold more blood to pump through the body or by becoming relatively stiff and/or thickened (hypertrophy). The walls of heart 10 can eventually weaken and become unable to pump as efficiently. In some cases, the kidneys can respond to cardiac inefficiency by causing the body to retain fluid. Fluid build-up in arms, legs, ankles, feet, lungs, and/or other organs can cause the body to become congested, which is referred to as congestive heart failure. Generally, left atrial pressure can be relatively highly correlated with risk of congestive heart failure. Furthermore, there can generally be a relatively strong correlation between increases in left atrial pressure and pulmonary congestion. Acute decompensated congestive heart failure is a leading cause of morbidity and mortality, and therefore early detection and treatment of congestive heart failure is a significant concern in medical care. Examples of the present disclosure can detect characteristics of the fluid in this cardiac space, enabling physicians to be better able to diagnose and treat these conditions. Specifically, cardiac pressure indicators can present weeks prior to hospitalization in some patients. Therefore, continuous recording and analysis of these pressures can advantageously be implemented as an early warning measure to reduce risks of hospitalization and/or the onset of heart failure.

In some implementations, the present disclosure relates to systems, devices, and methods for measuring a characteristic of fluid surrounding a cardiac device. The cardiac device can measure a pressure, a flow, a temperature, an oxygen saturation, glucose, or any other characteristics of a fluid surrounding the cardiac device and/or of heart 10 which the cardiac device is implanted into. The cardiac device can be implanted in an opening created between right pulmonary veins 50 and right atrium 12 or superior vena cava 44. In alternate examples, the cardiac device can be implanted between right pulmonary veins 50 and inferior vena cava 42. In further alternate examples, the cardiac device can be implanted between right pulmonary veins 50 and right pulmonary artery 46. In additional alternate examples, the cardiac device can be implanted between left pulmonary veins 52 and left pulmonary artery 48.

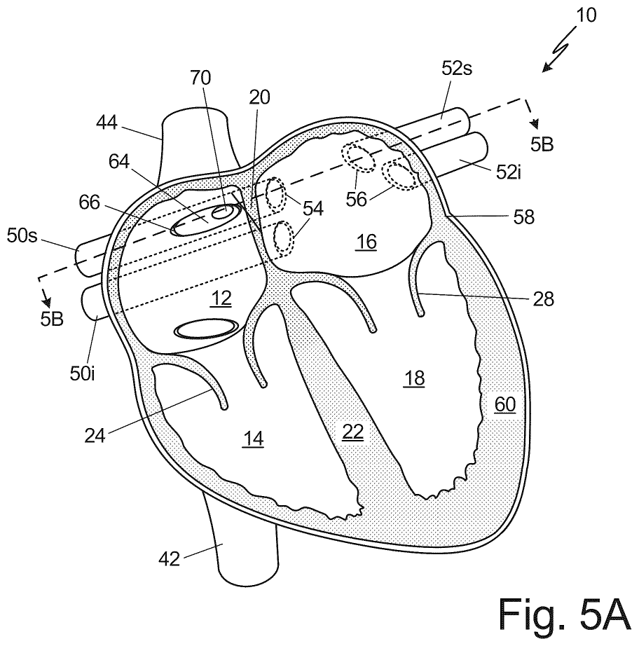

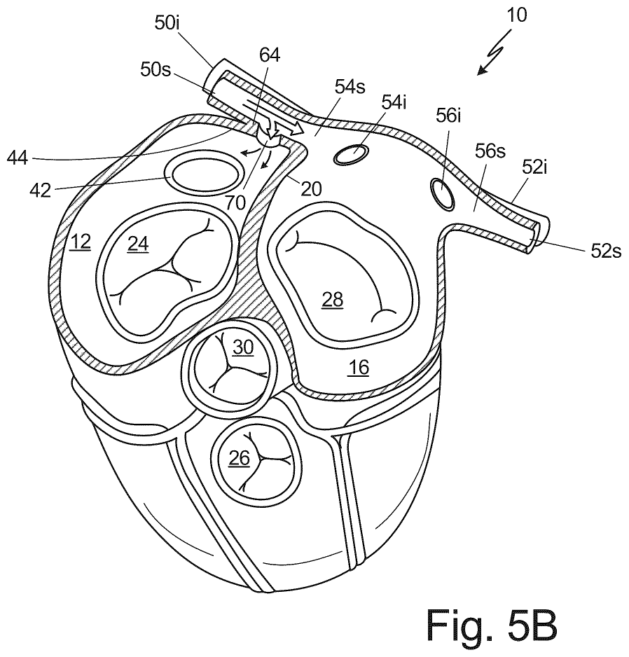

FIG. 5A is a frontal cross-sectional view of heart 10 having pulmonary vein opening 70. FIG. 5B is a superior cross-sectional view of heart 10, taken along line 5B-5B of FIG. 5A, having pulmonary vein opening 70. FIGS. 5A and 5B will be discussed together. FIGS. 5A-5B show heart 10, right atrium 12, right ventricle 14, left atrium 16, left ventricle 18, atrial septum 20, ventricle septum 22, tricuspid valve 24, pulmonary valve 26, mitral valve 28, aortic valve 30, inferior vena cava 42, superior vena cava 44, right pulmonary veins 50 (including right inferior pulmonary vein 50i and right superior pulmonary vein 50s), left pulmonary veins 52 (including left inferior pulmonary vein 52i and left superior pulmonary vein 52s), right pulmonary vein ostia 54 (including right inferior pulmonary vein ostium 54i and right superior pulmonary vein ostium 54s), left pulmonary vein ostia 56 (including left inferior pulmonary vein ostium 56i and left superior pulmonary vein ostium 56s), base 58, apex 60, common wall 64, superior vena cava ostium 66, and opening 70.

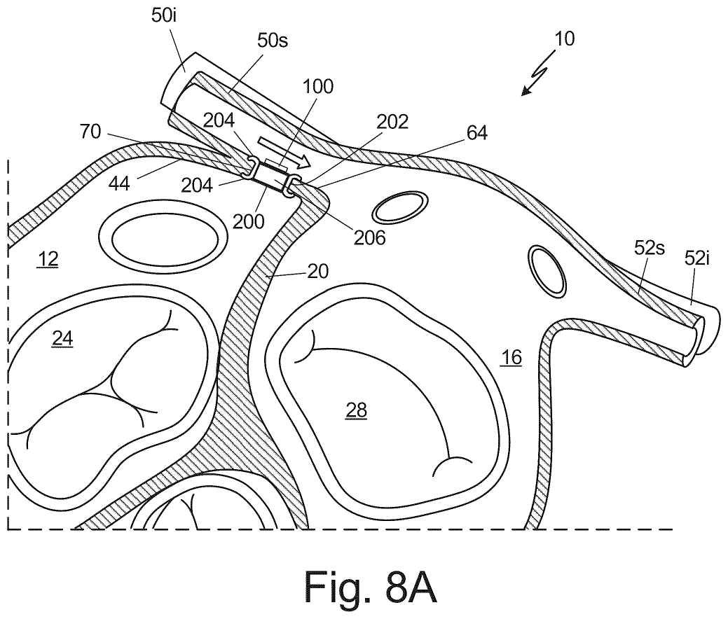

Heart 10 has the structure as described above in reference to FIGS. 1-4. As shown in FIGS. 5A-5B, heart 10 includes common wall 64 between right pulmonary veins 50 and superior vena cava 44. In some patients, the anatomy of right superior pulmonary vein 50s is positioned and oriented such that common wall 64 between right superior pulmonary vein 50s and superior vena cava 44 can be present, such as at or near superior vena cava ostium 66 of superior vena cava 44 that opens into right atrium 12. Opening 70 can be formed in common wall 64 of heart 10. Once opening 70 is created, a cardiac device can be implanted therein, as will be discussed in more detail below with respect to FIGS. 7-18. Therefore, a cardiac device positioned in opening 70 of common wall 64 can permit for pressure measurement of both right atrium 12 (either directly or via superior vena cava 44) and left atrium 16 (via right superior pulmonary vein 50s) without traversing atrial septum 20.

Common wall 64 can be present near the septal boundary between right atrium 12 and left atrium 16. For example, right superior pulmonary vein ostium 54s of right superior pulmonary vein 50s can likewise be positioned in an area that is relatively close to atrial septum 20, wherein an area near right superior pulmonary vein ostium 54s within right superior pulmonary vein 50s on a generally anterior side of right superior pulmonary vein 50s can be shared with the wall of superior vena cava 44 and/or right atrium 12.

Although certain examples are described herein as including or involving opening 70 formed in a wall separating right superior pulmonary vein 50s from right atrium 12, in some patients, a suitable shared wall area between right superior pulmonary vein 50s and the right side of heart 10 can be below superior vena cava ostium 66, such that opening 70 opens directly into right atrium 12 rather than into superior vena cava 44. Therefore, description herein of cardiac devices between right superior pulmonary vein 50s and superior vena cava 44 should be understood to also relate to and disclose cardiac devices between right superior pulmonary vein 50s and right atrium 12 below superior vena cava ostium 66.

Opening 70 can be formed using a transcatheter delivery system including one or more tools configured to form an opening in common wall 64. For example, opening 70 can be formed using an ablation tool, such as ultrasonic and/or radio-frequency radiation ablation tool or similar. Such ablation tool can be configured to burn the tissue of common wall 64 to cause opening 70 to be formed therein through the application of ultrasonic energy and/or radiofrequency radiation, or the like, emitted using an energy transducer component. For example, cardiac tissue ablation can be implemented by delivering energy, such as ultrasound or radiofrequency electromagnetic radiation energy, through a catheter or other transducer device to the target area of the pulmonary vein wall. Such energy can ablate or destroy relatively small focal areas of the cardiac tissue and form an opening therein. Ablation can further be implemented to cauterize the tissue around opening 70 formed in the pulmonary vein wall using other means (e.g., needle/wire, cutting blade, and/or dilator). In addition to ultrasound and radiofrequency radiation ablation, other types of catheter ablation can be implemented. For example, cardiac tissue ablation can be implemented using cryoablation, which generally utilizes a pressurized refrigerant in a catheter tip or other device to ablate the target tissue. According to some solutions, ablation can be implemented using minimally invasive techniques, such as through transcatheter access to the target atrium and/or other area of heart 10. Implantation of a cardiac device in accordance with examples of the present disclosure can be particularly desirable with respect to patients suffering from elevated left-side cardiac pressures, which generally are considered a high-risk patient demographic.

Additional solutions can be implemented for producing opening 70 in common wall 64. Such mechanism(s) can advantageously be implemented using transcatheter procedures. Example tools that can be implemented to form pulmonary vein opening 70 in tissue walls in accordance with aspects of the present disclosure can include one or more blades, needles, wires, and/or other devices having a relatively sharp point or edge and configured to be penetrated/cut through a cardiac tissue wall. In some implementations, a cutting tool configured to excise a cut-out portion of the tissue wall and remove the cut-out portion of tissue using the delivery system can be used to form the opening 70.

The implementation of opening 70 from within right superior pulmonary vein 50s can provide certain advantages or benefits relative to certain other opening 70 positions/locations. For example, the position within right pulmonary vein 50 can provide a position relatively far away from mitral valve 28, such that a cardiac device can be relatively far from mitral valve 28 and less inclined to obstruct flow within left atrium 16. Furthermore, the size of right pulmonary vein 50 can be of sufficient diameter to permit catheter access for the purpose of forming opening 70 and deploying a cardiac device therein.

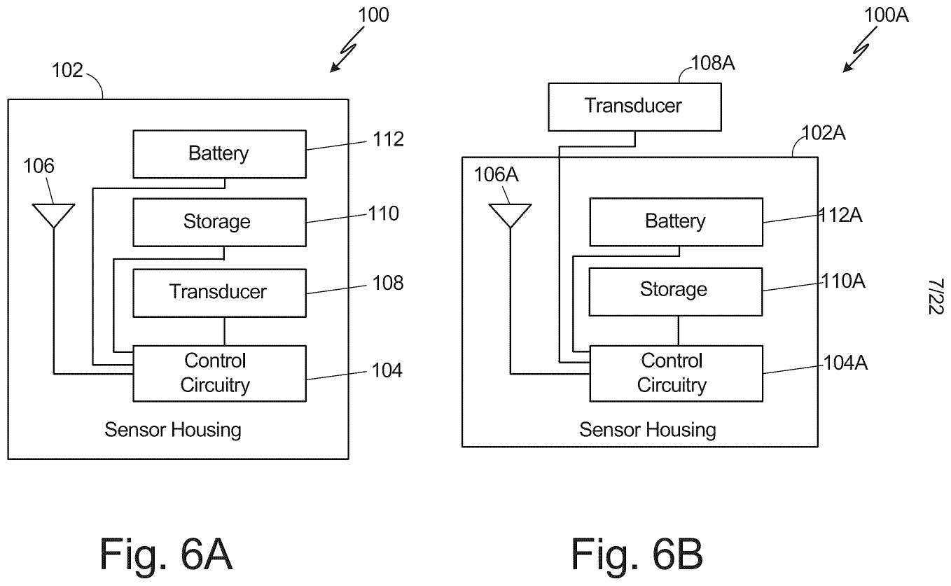

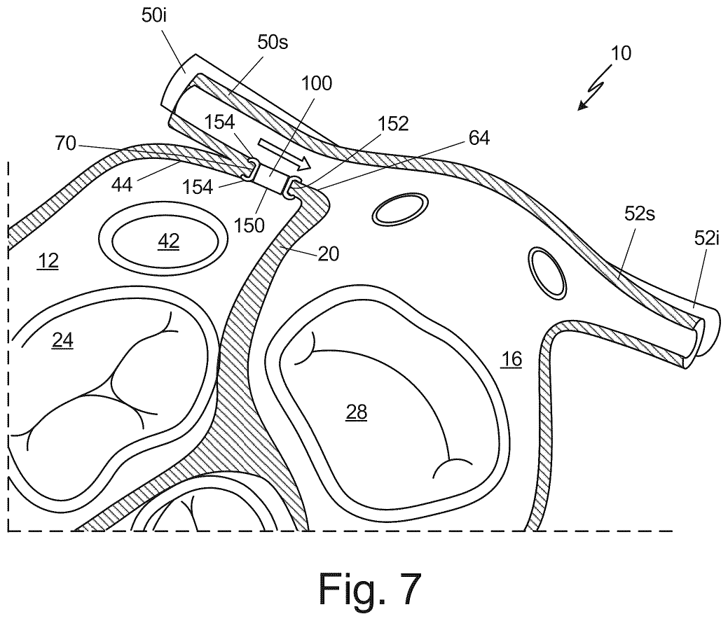

FIG. 6A is a block diagram of sensor device 100. FIG. 6B is a block diagram of sensor device 100A. FIGS. 6A and 6B will be discussed together. FIG. 6A shows sensor device 100, housing 102, control circuitry 104, antenna 106, transducer 108, storage 110, and battery 112. FIG. 6B shows sensor device 100A, housing 102A, control circuitry 104A, antenna 106A, transducer 108A, storage 110A, and battery 112A. FIG. 6A is an example of sensor device 100 with transducer 108 within sensor housing 102. FIG. 6B is an example of sensor device 100A with transducer 108A outside sensor housing 102A.

As shown in FIG. 6A, sensor device 100 includes sensor housing 102, control circuitry 104, antenna 106, transducer 108, storage 110, and battery 112. Sensor housing 102 hermetically seals control circuitry 104, antenna 106, transducer 108, storage 110, and battery 112 from the environment of heart 10. Sensor housing 102 can be a rigid body formed of stainless steel or another rigid biocompatible material. Alternatively, sensor housing 102 can be a flexible body formed of a biocompatible polymer.

Control circuitry 104 communicatively and/or electrically connects antenna 106, transducer 108, storage 110, and battery 112. Control circuitry 104 will access programming on storage 110 and carry out the programming. Control circuitry 104 can send a signal to transducer 108 to sense a signal from the fluid surrounding heart 10 and/or from heart 10. The signal sensed by transducer 108 can be communicated back to control circuitry 104. The signal can then be communicated to storage 110 for storage or to antenna 106. Antenna 106 enables connection from sensor device 100 to a secondary system, for example an external system positioned outside of the body. Antenna 106 can transmit data and/or electrical power. When antenna 106 transmits data, control circuitry 104 will establish a data connection between storage 110 and the secondary system via antenna 106. When the connection transmits electrical power, control circuitry 104 will establish an electrical connection between antenna 106 and battery 112.

Control circuitry 104, in one example, is configured to implement functionality and/or process instructions. For example, control circuitry 104 can be capable of processing instructions stored in storage 110. Examples of control circuitry 104 can include one or more of a processor, a microprocessor, a controller, a digital signal processor (DSP), an application specific integrated circuit (ASIC), a field-programmable gate array (FPGA), or other equivalent discrete or integrated logic circuitry. Control circuitry 104 can be entirely or partially mounted on one or more circuit boards.

Storage 110 of sensor device 100 can be configured to store data and information before, during, and/or after operation. For example, storage 110 can store instructions that can be executed by control circuitry 104. Further, storage 110 can store data collected by transducer 108. Storage 110, in some examples, is described as memory or computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, storage 110 is a temporary memory, meaning that a primary purpose of the memory is not long-term storage. Storage 110, in some examples, is described as volatile memory, meaning that storage 110 does not maintain stored contents when power to sensor device 100 is turned off. Examples of volatile memories can include random access memories (RAM), dynamic random access memories (DRAM), static random access memories (SRAM), and other forms of volatile memories. In some examples, storage 110 is used to store program instructions for execution by control circuitry 104. Storage 110, in one example, is used by software or applications running on sensor device 100 to temporarily store information during program execution.

Storage 110, in some examples, also includes one or more computer-readable storage media. Storage 110 can be configured to store larger amounts of information than volatile memory. Storage 110 can further be configured for long-term storage of information. In some examples, storage 110 includes non-volatile storage elements. Examples of such non-volatile storage elements can include magnetic hard discs, optical discs, flash memories, or forms of electrically programmable memories (EPROM) or electrically erasable and programmable (EEPROM) memories.

Transducer 108 can be any transducer (or sensor) that is capable of sensing a signal from the blood in and around heart 10 and/or from heart 10. The signal sensed by transducer 108 is representative of or a direct measurement of a characteristics of the blood in and around heart 10 and/or heart 10. Transducer 108 can, for example, be a pressure sensor, a flow sensor, a temperature sensor, an oxygen saturation sensor, a glucose sensor, or any other suitable sensor. In some examples, transducer 108 can be a piezoelectric transducer, a strain gauge, or a fiber optic pressure sensor.

Battery 112 stores electrical power and powers control circuitry 104, antenna 106, transducer 108, and storage 110. Battery 112 can be wirelessly recharged by antenna 106. Alternatively, battery 112 can be non-rechargeable. Battery 112 can be any suitable battery known to those of skill in the art as being able to power medical devices.

As shown in FIG. 6B, sensor device 100A includes sensor housing 102A, control circuitry 104A, antenna 106A, transducer 108A, storage 110A, and battery 112A. Sensor device 100A has generally the same structure, design, and function as sensor device 100 shown in FIG. 6A, however transducer 108A is positioned outside of sensor housing 102A. If transducer 108A is outside of sensor housing 102A as illustrated in FIG. 6B, transducer 108A can collect data regarding the characteristics of the fluid which transducer 108A is immersed, including for example pressure, flow, temperature, oxygen saturation, glucose, or any other characteristic that one of skill in the art could measure from blood.

In an alternate example, a sensor device can comprise transducers both within sensor housing 102 and outside of sensor housing 102. In the following discussion of FIGS. 7-18, reference numeral 100 will be used to refer to a sensor device that has a transducer positioned in sensor housing 102 and/or outside of sensor housing 102.

The internal components of sensor device 100 described above in reference to FIGS. 6A-6B are intended to be exemplary. Sensor device 100 can include more, less, or other suitable components.

FIG. 7 is a superior cross-sectional view of heart 10 having cardiac device 150 with sensor 100 implanted therein. FIG. 7 shows heart 10, which includes right atrium 12, left atrium 16, atrial septum 20, tricuspid valve 24, mitral valve 28, inferior vena cava 42, superior vena cava 44, right pulmonary veins 50 (including right inferior pulmonary vein 50i and right superior pulmonary vein 50s), left pulmonary veins 52 (including left inferior pulmonary vein 52i and left superior pulmonary vein 52s), common wall 64, and opening 70. FIG. 7 further show cardiac device 150, which includes body 152, anchor arms 154, and sensor 100.

FIG. 7 shows heart 10, as described above in reference to FIGS. 1-5B. FIG. 7 further shows cardiac device 150, which is one example of a cardiac device that can be positioned in opening 70 of common wall 64. Cardiac device 150 includes body 152 that extends through opening 70. Anchor arms 154 extend outward from a first end and a second end of body 152 and are configured to anchor cardiac device 150 in opening 70. Sensor 100 is housed in body 152 of cardiac device 150. Body 152 is one example of sensor housing 102 that houses and hermetically seals the components of sensor 100 into body 152, including control circuitry 104, antenna 106, transducer 108, storage 110, and/or battery 112 as discussed above in reference to FIGS. 6A-6B. Body 152 can have any suitable or desirable cross-sectional shape or area, such as circular, oblong, ovoid, elliptical, rectangular, or any other shape. In some examples, body 152 of cardiac device 150 is at least partially rigid, such that the form thereof may be substantially maintained over time after implantation.

Cardiac device 150 can be implanted to enable continuous measurement of characteristics of fluid surrounding cardiac device 150 and/or heart 10. Sensor 100 of cardiac device 150 can measure a pressure, a flow, a temperature, an oxygen saturation, a glucose, or any other characteristic of a fluid (for example, blood) surrounding cardiac device 150 and/or of heart 10 which cardiac device 150 is implanted into. Cardiac device 150 can be implanted in opening 70 created between right superior pulmonary vein 50s and right atrium 12 or superior vena cava 44. When cardiac device 150 is implanted into opening 70, a first end of cardiac device 150 will be positioned in right superior pulmonary vein 50s, and a second end of cardiac device 150 will be positioned in right atrium 12 or superior vena cava 44. When a first end of cardiac device 150 is positioned in right superior pulmonary vein 50s, sensor 100 can sense a characteristic of the blood in right superior pulmonary vein 50s. Due to the proximity and connectivity of right superior pulmonary vein 50s and left atrium 16, the characteristics of the blood in right superior pulmonary vein 50s will be substantially similar to the characteristics of the blood in left atrium 16. When a second end of cardiac device 150 is positioned in right atrium 12, sensor 100 can sense a characteristic of the blood in right atrium 12. When a second end of cardiac device 150 is positioned in superior vena cava 44, sensor 100 can sense a characteristic of the blood in superior vena cava 44. Due to the proximity and connectivity of superior vena cava 44 and right atrium 12, the characteristics of the blood in superior vena cava 44 will be substantially similar to the characteristics of the blood in right atrium 12.

In one example, sensor 100 can be a pressure sensor and can be used to sense a pressure in right superior pulmonary vein 50s and/or a pressure in right atrium 12 or superior vena cava 44. The pressure of the blood in right superior pulmonary vein 50s is the same as the pressure in left atrium 16, thus the pressure sensed in right superior pulmonary vein 50s can be used as a direct measurement of the pressure of the blood in left atrium 16.

Studies have shown that episodes of heart failure decompensation requiring hospitalization are typically preceded by a rise in left atrial pressure. Left atrial pressure is a direct reflection of left ventricular filling pressure, which is the primary target for congestive heart failure management. Left atrial pressure is typically estimated by measuring pulmonary artery pressure using a pulmonary artery catheter (or Swan-Ganz catheter). A direct measurement of left atrial pressure allows for more accurate clinical information than an estimation based on pulmonary artery pressure.

Cardiac device 150 allows for real-time continuous monitoring of left atrial pressure. Cardiac device 150 allows for direct measurement of left atrial pressure via the measurement of pressure in right superior pulmonary vein 50s. The direct measurement of left atrial pressure can assist clinicians in early diagnosis of heart failure decompensation. The placement of cardiac device 150 in common wall 64 between right superior pulmonary vein 50s and right atrium 12 or superior vena cava 44 also maintains atrial septum 20 for future transseptal procedures. Further, the placement of cardiac device 150 in common wall 64 between right superior pulmonary vein 50s and right atrium 12 or superior vena cava 44 prevents cardiac device 150 from interfering with mitral valve 28.

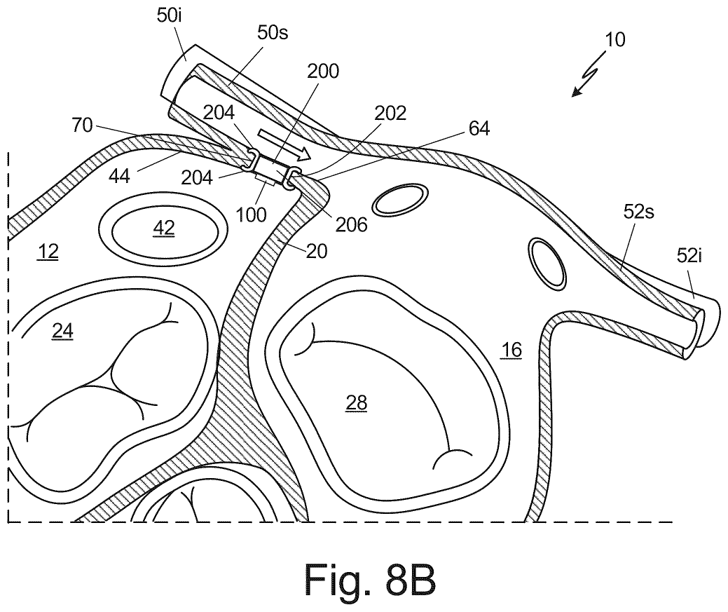

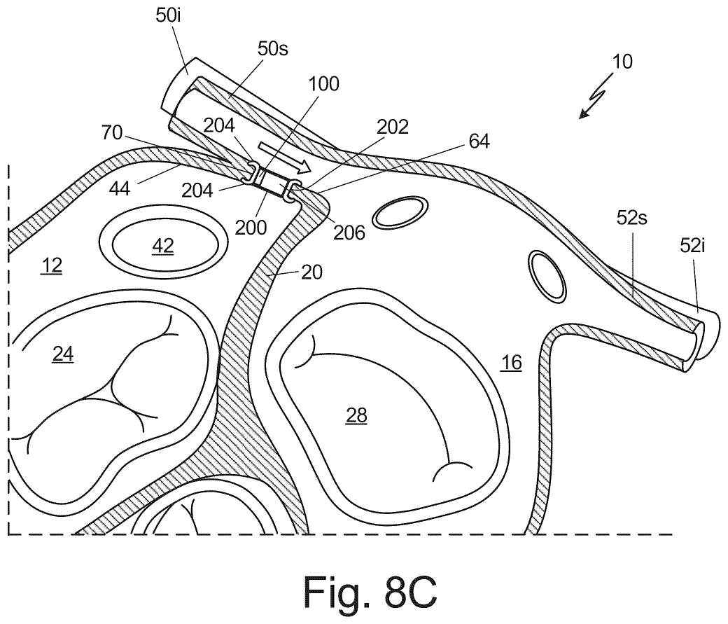

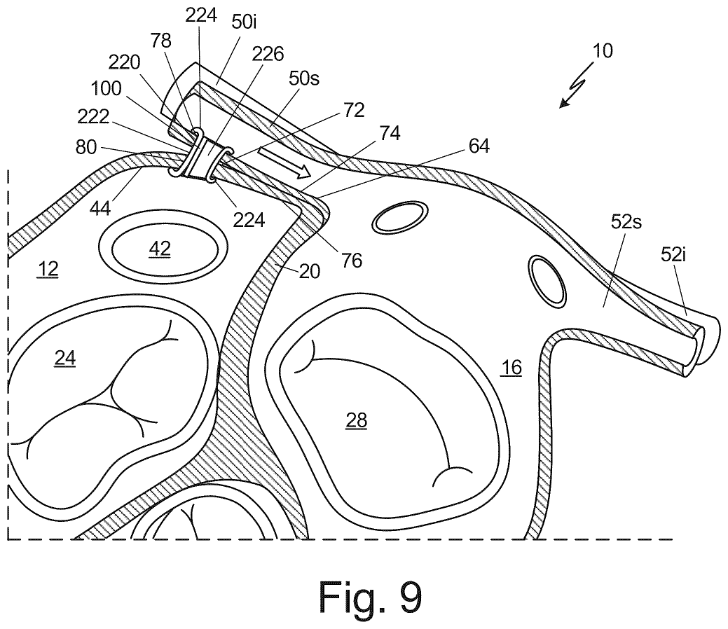

FIG. 8A is a superior cross-sectional view of heart 10 having cardiac device 200 with sensor 100 on a first end implanted therein. FIG. 8B is a superior cross-sectional view of heart 10 having cardiac device 200 with sensor 100 on a second end implanted therein. FIG. 8C is a superior cross-sectional view of heart 10 having cardiac device 200 with sensor 100 extending through cardiac device 200 implanted therein. FIGS. 8A-8C will be discussed together. FIGS. 8A-8C show heart 10, which includes right atrium 12, left atrium 16, atrial septum 20, tricuspid valve 24, mitral valve 28, inferior vena cava 42, superior vena cava 44, (including right inferior pulmonary vein 50i and right superior pulmonary vein 50s), left pulmonary veins 52 (including left inferior pulmonary vein 52i and left superior pulmonary vein 52s), common wall 64, and opening 70. FIGS. 8A-8C further show cardiac device 200, which includes body 202, anchor arms 204, opening 206, and sensor 100.

FIGS. 8A-8C show heart 10, as described above in reference to FIGS. 1-5B. FIGS. 8A-8C further show cardiac device 200, which is one example of a cardiac device that can be positioned in opening 70 of common wall 64. Cardiac device 200 is a shunt device in the example shown in FIGS. 8A-8C. Cardiac device 200 includes body 202 that extends through opening 70. Body 202 can have a stent structure that is formed of struts and openings. Anchor arms 204 extend outward from a first end and a second end of body 202 and are configured to anchor cardiac device 200 in opening 70. Opening 206 extends through body 202 and fluidly couples right superior pulmonary vein 50s to right atrium 12 or superior vena cava 44. Sensor 100 is attached to cardiac device 200.

FIG. 8A shows cardiac device 200 with sensor 100 attached to a first end of cardiac device 200 so sensor 100 is configured to be positioned in right superior pulmonary vein 50s. When sensor 100 is attached to the first end of cardiac device 200, it is positioned to sense signals from blood flowing through right superior pulmonary vein 50s. FIG. 8B shows cardiac device 200 with sensor 100 attached to a second end of cardiac device 200 so sensor 100 is configured to be positioned in right atrium 12 or superior vena cava 44. When sensor 100 is attached to the second end of cardiac device 200, it is positioned to sense signals from blood flowing through right atrium 12 or superior vena cava 44. FIG. 8C illustrates cardiac device 200 with sensor 100 extending through opening 206 of body 202 of cardiac device 200. When sensor 100 extends through body 202 of cardiac device 200, signals can be sensed from blood flowing through right superior pulmonary vein 50s and from blood flowing through right atrium 12 or superior vena cava 44. Sensor 100 can be anchored to body 202 and/or anchor arms 204 of cardiac device 200 using any suitable means and in any suitable position. Cardiac device 200 can have any suitable or desirable form or shape.

In the example shown in FIGS. 8A-8C, cardiac device 200 is configured to be a shunt device capable of shunting blood between right superior pulmonary vein 50s and right atrium 12 or superior vena cava 44. When cardiac device 200 is a shunt device, blood can be shunted from left atrium 16 (via right superior pulmonary vein 50s) to right atrium 12 (either directly or via superior vena cava 44). Shunting blood from left atrium 16 to right atrium 12 can reduce pressure on the left side of heart 10. In alternate examples, cardiac device 200 is not a shunt device.

Cardiac device 200 can be implanted to enable continuous measurement of characteristics of fluid surrounding cardiac device 200 and/or heart 10. Sensor 100 of cardiac device 200 can measure a pressure, a flow, a temperature, an oxygen saturation, a glucose, or any other characteristic of a fluid surrounding cardiac device 200 and/or of heart 10 which cardiac device 200 is implanted into. Cardiac device 200 can be implanted in opening 70 created between right superior pulmonary vein 50s and right atrium 12 or superior vena cava 44. When cardiac device 200 is implanted into opening 70, a first end of cardiac device 200 will be positioned in right superior pulmonary vein 50s, and a second end of cardiac device 200 will be positioned in right atrium 12 or superior vena cava 44. When a first end of cardiac device 200 is positioned in right superior pulmonary vein 50s and sensor 100 is positioned on the first end of or extending through cardiac device 200, sensor 100 can sense a characteristic of the blood in right superior pulmonary vein 50s. Due to the proximity and connectivity of right superior pulmonary vein 50s and left atrium 16, the characteristics of the blood in right superior pulmonary vein 50s will be substantially similar to the characteristics of the blood in left atrium 16. When a second end of cardiac device 200 is positioned in right atrium 12 and sensor 100 is positioned on a second end of or extending through cardiac device 200, sensor 100 can sense a characteristic of the blood in right atrium 12. When a second end of cardiac device 200 is positioned in superior vena cava 44 and sensor 100 is positioned on a second end of or extending through cardiac device 200, sensor 100 can sense a characteristic of the blood in superior vena cava 44. Due to the proximity and connectivity of superior vena cava 44 and right atrium 12, the characteristics of the blood in superior vena cava 44 will be substantially similar to the characteristics of the blood in right atrium 12.

In one example, sensor 100 can be a pressure sensor and can be used to sense a pressure in right superior pulmonary vein 50s and/or a pressure in right atrium 12 or superior vena cava 44. The pressure of the blood in right superior pulmonary vein 50s is the same as the pressure in left atrium 16, thus the pressure sensed in right superior pulmonary vein 50s can be used as a direct measurement of the pressure of the blood in left atrium 16.

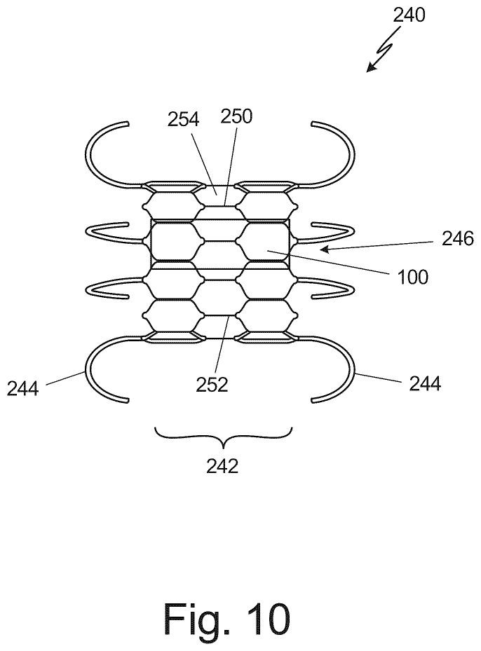

FIG. 9 is a superior cross-sectional view of heart 10 having cardiac device 220 with sensor 100 extending through cardiac device 220 implanted therein. FIG. 9 shows heart 10, which includes right atrium 12, left atrium 16, atrial septum 20, tricuspid valve 24, mitral valve 28, inferior vena cava 42, superior vena cava 44, right pulmonary veins 50 (including right inferior pulmonary vein 50i and right superior pulmonary vein 50s), left pulmonary veins 52 (including left inferior pulmonary vein 52i and left superior pulmonary vein 52s), common wall 64, gap 72, pulmonary vein wall 74, right atrium wall 76, opening 78, and opening 80. FIG. 9 further shows cardiac device 220, which includes body 222, anchor arms 224, opening 226, and sensor 100.

FIG. 9 shows heart 10, as described above in reference to FIGS. 1-5B. In the example of heart 10 shown in FIG. 9, common wall 64 between right superior pulmonary vein 50s and right atrium 12 or superior vena cava 44 is very small, and gap 72 extends between pulmonary vein wall 74 of right superior pulmonary vein 50s and right atrium wall 76 of right atrium 12. In some patients, sufficient area of common wall 64 may not be present. As such, implanting cardiac device 220 in an area that is not in common wall 64 between right superior pulmonary vein 50s and superior vena cava 44 or right atrium 12 can be desirable. Therefore, in some examples, cardiac device 220 can be implanted in a manner as to be between right superior pulmonary vein 50s and superior vena cava 44 or right atrium 12, wherein the walls associated with such vessel/chambers in the area of cardiac device 220 are not common, but rather separate walls that contact one another and/or are separated by gap 72 in one or more areas. For example, as shown in FIG. 9, right superior pulmonary vein 50s can have pulmonary vein wall 74 on a side of right superior pulmonary vein 50s oriented towards the target right-side vessel/chamber, wherein right atrium wall 76 of right atrium 12 is separate from pulmonary vein wall 74 in the area of cardiac device 220. In alternative examples, right atrium wall 76 can be a wall of superior vena cava 44. In at least some areas, gap 72 can be present between opposing walls of pulmonary vein wall 74 and right atrium wall 76.

Cardiac device 220 has a similar structure as cardiac device 200 shown in FIGS. 8A-8C above, however cardiac device 220 has an elongated body 302 that spans gap 72. Opening 78 can be formed in pulmonary vein wall 74, and opening 80 can be formed in right atrium wall 76. Body 222 of cardiac device 220 can be positioned in opening 78 and opening 80. Body 222 can have a stent structure that is formed of struts and openings. Anchor arms 224 extend outward from a first end and a second end of body 222 and are configured to anchor cardiac device 220 in opening 78 and opening 80. Anchor arms 224 on the first end of body 222 anchor cardiac device 220 to pulmonary vein wall 74, and anchor arms 224 on the second end of body 222 anchor cardiac device 220 to right atrium wall 76. Opening 226 extends through body 222 and fluidly couples right superior pulmonary vein 50s to right atrium 12 or superior vena cava 44. Sensor 100 is attached to cardiac device 220. In the example shown in FIG. 9, sensor 100 extends through opening 226 in body 222 of cardiac device 220 and is configured to sense signals from blood in right superior pulmonary vein 50s and right atrium 12 or superior vena cava 44. In an alternate example, sensor 100 can be attached to a first end of cardiac device 220 or to a second end of cardiac device 220, similar to the examples of cardiac device 200 shown in FIGS. 8A-8B discussed above.

In the example shown in FIG. 9, cardiac device 220 is configured to be a shunt device capable of shunting blood between right superior pulmonary vein 50s and right atrium 12 or superior vena cava 44. When cardiac device 220 is a shunt device, blood can be shunted from left atrium 16 (via right superior pulmonary vein 50s) to right atrium 12 (either directly or via superior vena cava 44). Shunting blood from left atrium 16 to right atrium 12 can reduce pressure on the left side of heart 10. When cardiac device 220 is formed in an area where there is no common wall between right superior pulmonary vein 50s and superior vena cava 44 or right atrium 12 and when cardiac device 220 has a stent structure, body 222 can have a cover to prevent blood leakage outside of heart 10 into the chest cavity through body 222. For example, cardiac device 220 can advantageously be fluid-tight at least where the cover is present to ensure that blood does not leak through body 222. In some examples, the first end and the second end of cardiac device 220 can include flanges to prevent blood leaking around anchor arms 224. For example, anchor arms 224 can be secured to the respective interior cardiac walls in a manner as to prevent blood from leaking under anchor arms 224 and out around the cover. In alternate examples, cardiac device 200 is not a shunt device.

Cardiac device 220 can be implanted to enable continuous measurement of characteristics of fluid surrounding cardiac device 220 and/or heart 10. Sensor 100 of cardiac device 220 can measure a pressure, a flow, a temperature, an oxygen saturation, a glucose, or any other characteristic of a fluid surrounding cardiac device 220 and/or of heart 10 which cardiac device 220 is implanted into. Cardiac device 220 can be implanted in opening 70 created between right superior pulmonary vein 50s and right atrium 12 or superior vena cava 44. When cardiac device 220 is implanted into opening 70, a first end of cardiac device 220 will be positioned in right superior pulmonary vein 50s, and a second end of cardiac device 220 will be positioned in right atrium 12 or superior vena cava 44. When a first end of cardiac device 220 is positioned in right superior pulmonary vein 50s and sensor 100 is positioned on the first end of or extending through cardiac device 220, sensor 100 can sense a characteristic of the blood in right superior pulmonary vein 50s. Due to the proximity and connectivity of right superior pulmonary vein 50s and left atrium 16, the characteristics of the blood in right superior pulmonary vein 50s will be substantially similar to the characteristics of the blood in left atrium 16. When a second end of cardiac device 220 is positioned in right atrium 12 and sensor 100 is positioned on a second end of or extending through cardiac device 220, sensor 100 can sense a characteristic of the blood in right atrium 12. When a second end of cardiac device 220 is positioned in superior vena cava 44 and sensor 100 is positioned on a second end of or extending through cardiac device 220, sensor 100 can sense a characteristic of the blood in superior vena cava 44. Due to the proximity and connectivity of superior vena cava 44 and right atrium 12, the characteristics of the blood in superior vena cava 44 will be substantially similar to the characteristics of the blood in right atrium 12.

In one example, sensor 100 can be a pressure sensor and can be used to sense a pressure in right superior pulmonary vein 50s and/or a pressure in right atrium 12 or superior vena cava 44. The pressure of the blood in right superior pulmonary vein 50s is the same as the pressure in left atrium 16, thus the pressure sensed in right superior pulmonary vein 50s can be used as a direct measurement of the pressure of the blood in left atrium 16.

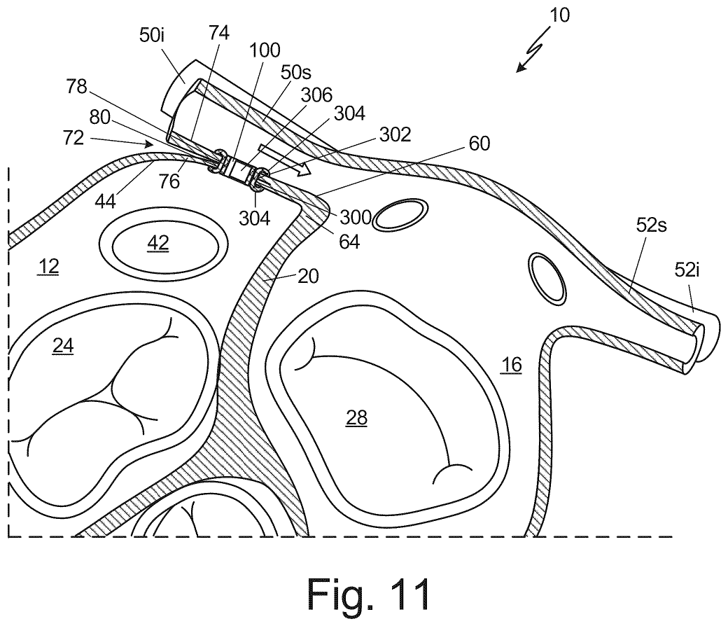

FIG. 10 is a side view of stent-type cardiac device 240 with sensor 100 extending through cardiac device 240. Cardiac device 240 includes body 242, anchor arms 244, opening 246, and sensor 100. Body 242 includes frame 250, which includes struts 252 and openings 254.

Cardiac device 240 is an example of a stent-type cardiac device. Cardiac device 240 provides an example of a structure that either cardiac device 200 shown in FIGS. 8A-8C or cardiac device 220 shown in FIG. 9 can take.

Cardiac device 240 includes body 242 that forms a central flow tube of cardiac device 240. Anchor arms 244 extend from a first end and a second end of body 242. Opening 246 extends through body 242. Cardiac device 240 is formed of frame 250, which includes a plurality of struts 252 and openings 254.

Cardiac device 240 can be an expandable or non-expandable implant device. In some examples, frame 250 of cardiac device 240 can be made out of a super elastic or shape-memory alloy that self-expands when released from a catheter. Although certain self-expanding frames and conduits are described herein as examples in some contexts, in some examples, cardiac device 240 can be balloon-expandable or may not require expansion after deployment. In some examples, cardiac device 240 is not collapsible and/or expandable. For example, cardiac device 240 can be implanted in a substantially deployed/expanded configuration in connection with a surgical or transcatheter (e.g., transfemoral) procedure or access to heart 10.

In the example shown in FIG. 10, anchor arms 244 are barb-or hook-type tissue anchors. In alternative examples, anchor arms 244 can be any shape known to those of skill in the art as being able to hook onto tissue of heart 10. Anchor arms 244 can project into heart 10, holding cardiac device 240 in place. In addition, or as an alternative, to barb-or hook-type tissue anchors, cardiac device 240 can have flanges or other anchor features which can be configured to hold cardiac device 240 in position within opening 70.

Anchor arms 244 as shown in FIG. 10, as well as those described in connection with other examples of the present disclosure, can be any suitable or desirable types of anchors. For example, in some examples, anchor arms 244 can be a pre-shaped wireform, such as a loop, coil, spiral, or the like, which can be configured to assume a relatively wide tissue anchor profile once deployed. Other types of anchor arms 244 that can be used include, but are not limited to, tension-fit or resistance-fit anchor arms 244. Anchor arms 244 can further include barb-type anchor arms 244, which can incorporate tip features configured to resist withdrawal of the anchor tip(s) from tissue in which the anchor tips are embedded following embedding. Anchor arms 244 can further include corkscrew-type anchors which are rotated into the tissue. Anchor arms 244 can further include other types of tissue anchors known to those of skill in the art as being able to anchor a medical device to heart 10.

Cardiac device 240 can have a length sufficient to traverse the distance from pulmonary vein wall 74 to right atrium wall 76. In some examples, cardiac device 240 can further include a cover and can be at least partially fluid-tight preventing fluid flow. The cover can be a thin polytetrafluoroethylene (PTFE) material, or other material or biological tissue. Anchor arms 244 can be anchored/implanted in the relevant biological tissue in any suitable or desirable way.

Sensor 100 is attached to an inside surface of body 242 of cardiac device 240. Sensor 100 can be secured to body 242 of cardiac device 240 using any suitable means. In some examples, sensor 100 can be integrally formed with body 242 of cardiac device 240. Sensor 100 is shown as extending through opening 246 of cardiac device 240, but can be attached to cardiac device 240 in any suitable location in alternate examples.

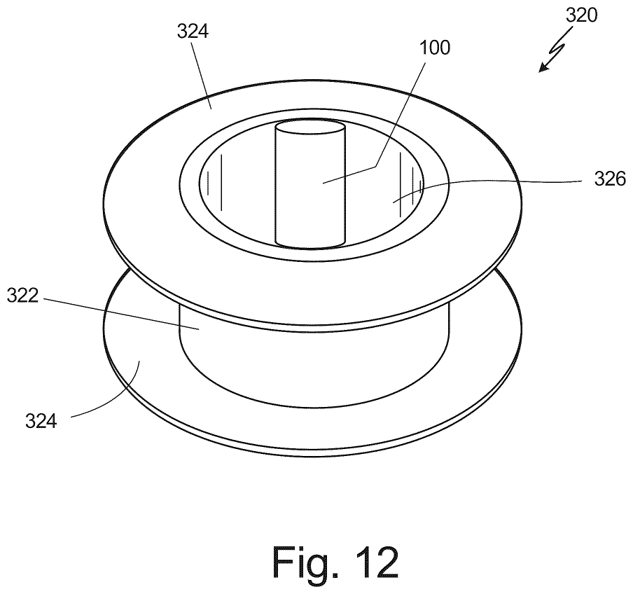

FIG. 11 is a superior cross-sectional view of heart 10 having cardiac device 300 implanted therein. FIG. 11 shows heart 10, which includes right atrium 12, left atrium 16, atrial septum 20, tricuspid valve 24, mitral valve 28, inferior vena cava 42, superior vena cava 44, right pulmonary veins 50 (including right inferior pulmonary vein 50i and right superior pulmonary vein 50s), left pulmonary veins 52 (including left inferior pulmonary vein 52i and left superior pulmonary vein 52s), common wall 64, gap 72, pulmonary vein wall 74, right atrium wall 76, opening 78, and opening 80. FIG. 11 further shows cardiac device 300, which includes body 302, anchor arms 304, opening 306, and sensor 100.

FIG. 11 shows heart 10, as described above in reference to FIGS. 1-5B. In the example of heart 10 shown in FIG. 11, common wall 64 between right superior pulmonary vein 50s and right atrium 12 or superior vena cava 44 is very small, and gap 72 extends between pulmonary vein wall 74 of right superior pulmonary vein 50s and right atrium wall 76 of right atrium 12 as described above with respect to FIG. 9. Therefore, as described above in connection with FIG. 9, implanting a cardiac device that spans gap 72 between pulmonary vein wall 74 and right atrium wall 76 can be desirable. In some cases, implanting cardiac device 300 is desirable, which is a grommet-type cardiac implant that permits pulmonary vein wall 74 and right atrium wall 76 to be clamped together can be desirable.

Cardiac device 300 has a similar structure as cardiac device 200 shown in FIGS. 8A-8C above, however cardiac device 300 is a grommet type cardiac implant. Opening 78 can be formed in pulmonary vein wall 74, and opening 80 can be formed in right atrium wall 76. Body 302 of cardiac device 300 can be positioned in opening 78 and opening 80. Anchor arms 304 extend outward from a first end and a second end of body 302 and are configured to anchor cardiac device 300 in opening 78 and opening 80. Anchor arms 304 on the first end of body 302 anchor cardiac device 300 to pulmonary vein wall 74, and anchor arms 304 on the second end of body 302 anchor cardiac device 300 to right atrium wall 76. Opening 306 extends through body 302 and fluidly couples right superior pulmonary vein 50s to right atrium 12 or superior vena cava 44. Sensor 100 is attached to cardiac device 300. In the example shown in FIG. 11, sensor 100 extends through opening 306 in body 302 of cardiac device 300 and is configured to sense signals from blood in right superior pulmonary vein 50s and right atrium 12 or superior vena cava 44. In an alternate example, sensor 100 can be attached to a first end of cardiac device 300 or to a second end of cardiac device 300, similar to the examples of cardiac device 200 shown in FIGS. 8A-8B discussed above.

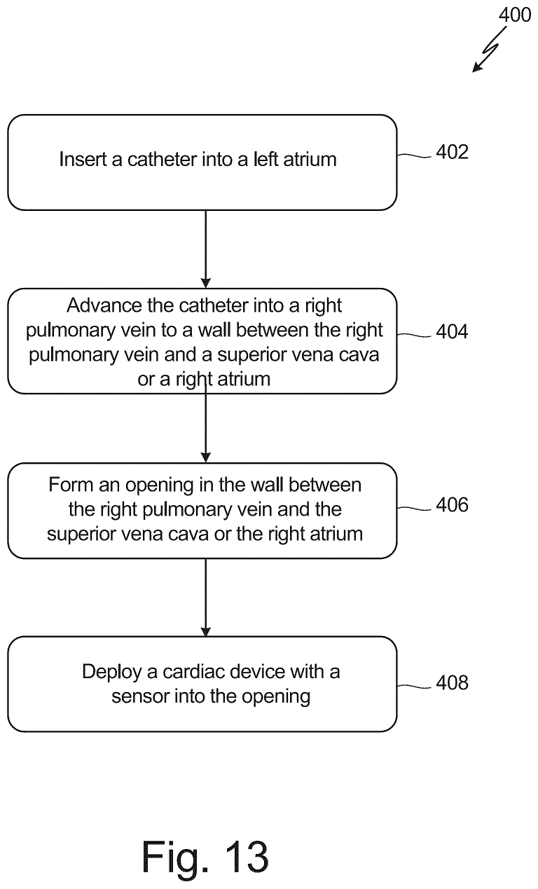

Cardiac device 300 is a grommet type cardiac device that permits pulmonary vein wall 74 and right atrium wall 76 to be clamped together. Such clamping can reduce the forces experienced by anchor arms 304 compared to body 202 of cardiac device 200, for example. Further, such clamping can standardize a length of body 302 of cardiac device 300. Cardiac device 300 can be at least partially rigid and include anchor arms 304 that are configured to clamp pulmonary vein wall 74 and right atrium wall 76 together, as will be discussed in more detail below with respect to FIG. 12.

As described, cardiac device 300 can serve to clamp pulmonary vein wall 74 and right atrium wall 76 together in order to provide a fluid-tight seal between the blood vessels/chambers and/or to secure cardiac device 300 in place. Furthermore, anchor arms 304 of cardiac device 300 as can serve to prevent or reduce tearing or abrasion of the tissue wall(s). As such, regardless of whether cardiac device 300 spans across common wall 64 or across pulmonary vein wall 74, gap 72, and right atrium wall 76, the structure of body 302 and anchor arms 304 can maintain the integrity of the tissue wall to prevent undesired tearing or other damage that can result in hemorrhage or other injury to the patient.

In some examples, cardiac device 300 can narrow or widen from right superior pulmonary vein 50s to superior vena cava 44 and/or right atrium 12. For example, in some examples, a first end of cardiac device 300 near right superior pulmonary vein 50s can have a first diameter that is different a second diameter of a second end near superior vena cava 44 and/or right atrium 12. Narrowing the first diameter can increase the compression fit of cardiac device 300 reducing a movement of cardiac device 300 after implantation. Further details relating to cardiac device 300 are described below in connection with FIG. 12.

In the example shown in FIG. 11, cardiac device 300 is configured to be a shunt device capable of shunting blood between right superior pulmonary vein 50s and right atrium 12 or superior vena cava 44. When cardiac device 300 is a shunt device, blood can be shunted from left atrium 16 (via right superior pulmonary vein 50s) to right atrium 12 (either directly or via superior vena cava 44). Shunting blood from left atrium 16 to right atrium 12 can reduce pressure on the left side of heart 10.

Cardiac device 300 can be implanted to enable continuous measurement of characteristics of fluid surrounding cardiac device 300 and/or heart 10. Sensor 100 of cardiac device 300 can measure a pressure, a flow, a temperature, an oxygen saturation, a glucose, or any other characteristic of a fluid surrounding cardiac device 300 and/or of heart 10 which cardiac device 300 is implanted into. Cardiac device 300 can be implanted in opening 70 created between right superior pulmonary vein 50s and right atrium 12 or superior vena cava 44. When cardiac device 300 is implanted into opening 70, a first end of cardiac device 300 will be positioned in right superior pulmonary vein 50s, and a second end of cardiac device 300 will be positioned in right atrium 12 or superior vena cava 44. When a first end of cardiac device 300 is positioned in right superior pulmonary vein 50s and sensor 100 is positioned on the first end of or extending through cardiac device 300, sensor 100 can sense a characteristic of the blood in right superior pulmonary vein 50s. Due to the proximity and connectivity of right superior pulmonary vein 50s and left atrium 16, the characteristics of the blood in right superior pulmonary vein 50s will be substantially similar to the characteristics of the blood in left atrium 16. When a second end of cardiac device 300 is positioned in right atrium 12 and sensor 100 is positioned on a second end of or extending through cardiac device 300, sensor 100 can sense a characteristic of the blood in right atrium 12. When a second end of cardiac device 300 is positioned in superior vena cava 44 and sensor 100 is positioned on a second end of or extending through cardiac device 300, sensor 100 can sense a characteristic of the blood in superior vena cava 44. Due to the proximity and connectivity of superior vena cava 44 and right atrium 12, the characteristics of the blood in superior vena cava 44 will be substantially similar to the characteristics of the blood in right atrium 12.

In one example, sensor 100 can be a pressure sensor and can be used to sense a pressure in right superior pulmonary vein 50s and/or a pressure in right atrium 12 or superior vena cava 44. The pressure of the blood in right superior pulmonary vein 50s is the same as the pressure in left atrium 16, thus the pressure sensed in right superior pulmonary vein 50s can be used as a direct measurement of the pressure of the blood in left atrium 16.

FIG. 12 is a perspective view of grommet-type cardiac device 320 with sensor 100 extending through cardiac device 320. Cardiac device 320 includes body 322, flanges 324, opening 326, and sensor 100.

Cardiac device 320 is an example of a grommet-type cardiac device. Cardiac device 320 provides an example of a structure that cardiac device 300 shown in FIG. 11 can take.

Cardiac device 320 includes body 322 that forms a central flow tube of cardiac device 320. Flanges 324 are anchor arms that extend from a first end and a second end of body 322. Opening 326 extends through body 322.

As described above with respect to FIG. 11, cardiac device 320 can be configured to clamp/secure pulmonary vein wall 74 to right atrium wall 76. Right atrium wall 76 can be a wall of right atrium 12 or of superior vena cava 44. Pulmonary vein wall 74 and right atrium wall 76 can be together such that they form common wall 64. Alternatively, pulmonary vein wall 74 and right atrium wall 76 can have gap 72 therebetween.

Cardiac device 320 can be inserted into opening 70. In the example shown in FIG. 12, flanges 324 can secure cardiac device 320 to one or more blood vessel/chamber wall(s). In some examples, flanges 324 can extend/project axially with respect to a central axis of cardiac device 320. As such, with flanges 324 retracted, cardiac device 320 can be substantially cylindrically shaped, however flanges 324 of cardiac device 320 can flare outwards to provide clamping/securing surfaces to keep cardiac device 320 in place. Flanges 324 can comprise shape-memory metal configured to assume the flared configuration as shown in FIG. 12 upon deployment from a delivery catheter or other delivery device/system. In some examples, when deployed, flanges 324 are angled at least partially towards one another to clamp or pinch the tissue wall(s) to which they are secured.

Sensor 100 is attached to an inside surface of body 322 of cardiac device 320. Sensor 100 can be secured to body 322 of cardiac device 320 using any suitable means. In some examples, sensor 100 can be integrally formed with body 322 of cardiac device 320. Sensor 100 is shown as extending through opening 326 of cardiac device 320, but an be attached to cardiac device 320 in any suitable location in alternate examples.

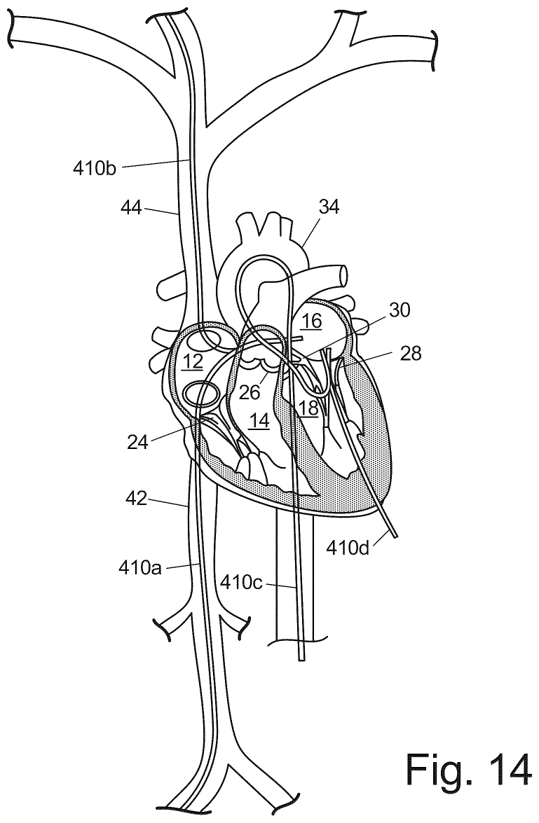

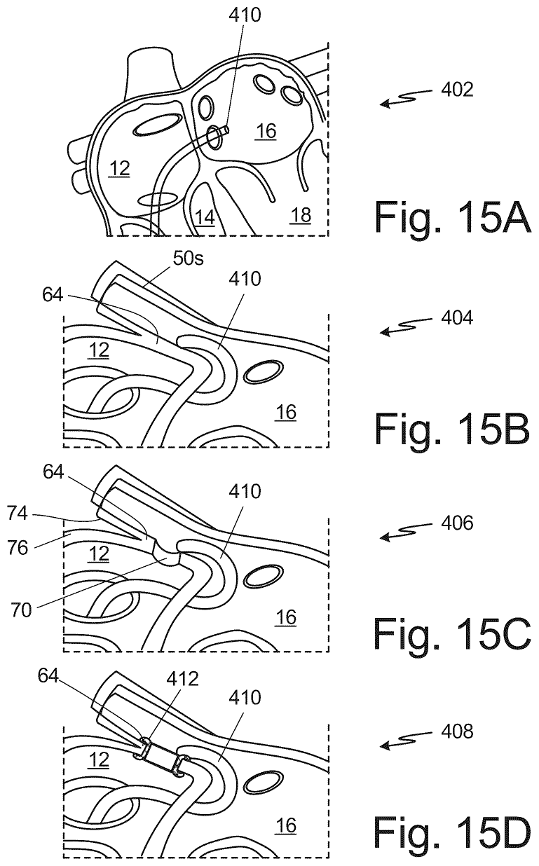

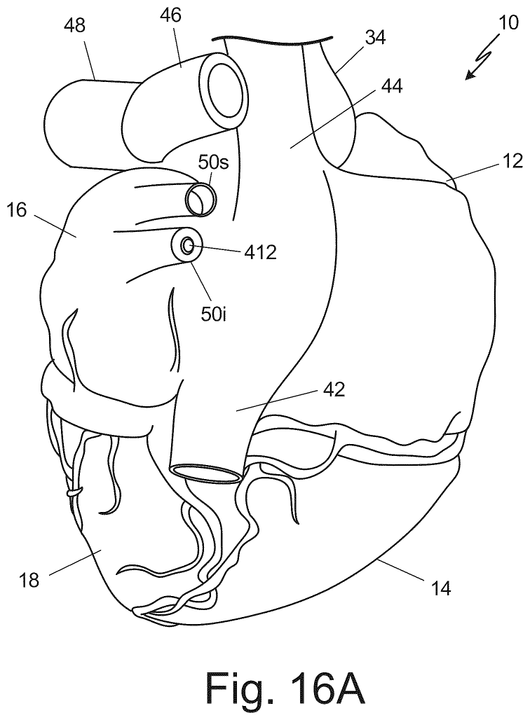

FIG. 13 is a flow diagram illustrating method 400 for forming pulmonary vein opening 70. FIG. 14 is a cutaway view of heart 10 and associated vasculature showing example catheter access paths for pulmonary vein procedures. FIGS. 15A-15D are cross-sectional views of heart 10 showing catheter 410 advancing through heart 10. FIGS. 13-15D will be discussed together. Method 400 includes steps 402, 404, 406, and 408.

In the discussion of FIGS. 13-15D, an example catheter is referred to as catheter 410. Catheter 410 can be any catheter that is suitable for forming an opening in a tissue wall and/or delivering a cardiac device to heart 10. Further, an example cardiac device is referred to as cardiac device 412. Cardiac device 412 can be any of cardiac devices 150, 200, 220, 240, 300, or 320 discussed above in reference to FIGS. 7-12. According to some solutions, cardiac device 412 implantation can be achieved using a transcatheter approach, wherein a catheter can be delivered to left atrium 16, and ultimately right superior pulmonary vein 50s, through the vasculature of the patient, such as through one or more arteries or veins of the arm, groin, or neck.

Step 402 includes inserting catheter 410 into left atrium 16. FIG. 14 is a cutaway view of heart 10 and associated vasculature showing certain catheter access paths for implantation procedures in accordance with one or more examples. FIG. 14 shows various catheters 410a-410d that can be used to implant cardiac device 412 in accordance with aspects of the present disclosure. Catheters 410a-410d can advantageously be steerable and relatively small in cross-sectional profile to permits traversal of the various blood vessels and chambers through which they can be advanced en route to, for example, left atrium 16 or other anatomy or chamber. Catheter access to left atrium 16 in accordance with certain implantation solutions can be made by first accessing right atrium 12 via inferior vena cava 42 (as shown by catheter 410a) or superior vena cava 44 (as shown by catheter 410b) and crossing atrial septum 20 to access left atrium 16.

Although access to left atrium 16 is illustrated and described in connection with certain examples as being through right atrium 12, inferior vena cava 42, and/or superior vena cava 44, such as through a transfemoral or other transcatheter procedure, other access paths/methods can be implemented in accordance with examples of the present disclosure. For example, in cases in which septal crossing through atrial septum 20 is not possible, other access routes can be taken to left atrium 16. In patients suffering from a weakened and/or damaged atrial septum 20, further engagement with atrial septum 20 can be undesirable and result in further damage to the patient. Furthermore, in some patients, atrial septum 20 can be occupied with one or more implant devices or other treatments, such that traversing atrial septum 20 is not tenable. As alternatives to transseptal access, transaortic access can be implemented, wherein catheter 410c is passed through aorta 34 and aortic valve 30, through left ventricle 18, and into left atrium 16 through mitral valve 28. Alternatively, transapical access can be implemented to access the target anatomy, as shown by delivery catheter 410d.

FIG. 15A shows one example of catheter 410 inserted into left atrium 16. Catheter 410 extends from inferior vena cava 42, through right atrium 12, through atrial septum 20, and into left atrium 16. FIG. 15A is one example of how catheter 410 can be inserted into left atrium 16.

Step 404 includes advancing catheter 410 into right superior pulmonary vein 50s to common wall 64 between right superior pulmonary vein 50s and superior vena cava 44 or right atrium 12. A distal end of catheter 410 is advanced to common wall 64 between right superior pulmonary vein 50s and superior vena cava 44 or right atrium 12. For example, common wall 64 can be a common wall between right superior pulmonary vein 50s and superior vena cava 44 or right atrium 12. Although shown as common wall 64, the wall portion targeted for opening 70 formation and cardiac device delivery can be a portion of pulmonary vein wall 74 that is separate from right atrium wall 76. As described above, right atrium wall 76 can alternatively or in addition include a wall of superior vena cava 44.

Step 406 includes forming opening 70 in common wall 64 between right superior pulmonary vein 50s and superior vena cava 44 or right atrium 12. Opening 70 can be formed in any suitable or desirable way, such as through wire/needle puncture, cutting, ablation, dilation, and or any combination thereof.

Step 408 includes deploying cardiac device 412 with sensor 100 in opening 70. Deploying cardiac device 412 can include inserting cardiac device 412 into opening 70 and expanding cardiac device 412. Expansion of cardiac device 412 can occur due to a deployment from catheter 410, due to a temperature change, or due to a force applied to cardiac device 412 by catheter 410. Deployment of cardiac device 412 can comprise expanding and/or inserting anchor arms and/or expanding flanges.

Various examples are described above relating to the placement of a cardiac device between right superior pulmonary vein 50s and right atrium 12 or superior vena cava 44. However, cardiac device placement in accordance with aspects of the present disclosure can involve deploying cardiac device between other pulmonary veins as well, wherein a cardiac device can be implanted between such other pulmonary vein(s) and a right-side chamber or vessel of the heart.

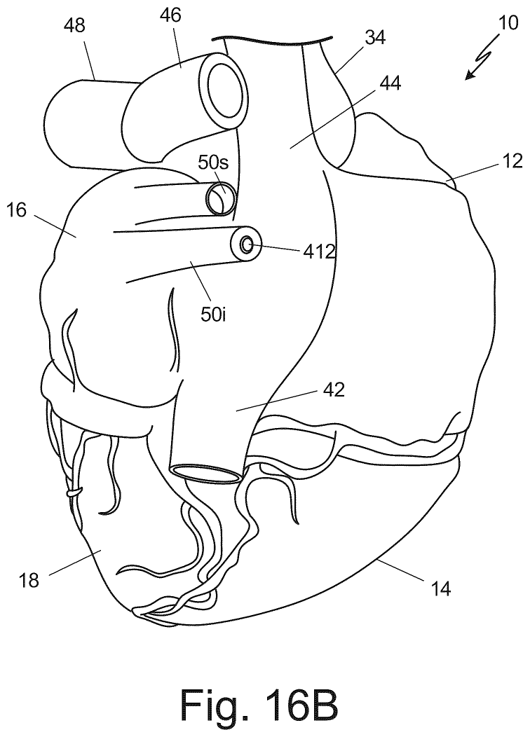

FIGS. 16A and 16B are posterior views of heart 10 having cardiac device 412 in right inferior pulmonary veins 50i. FIGS. 16A-16B show heart 10, right atrium 12, right ventricle 14, left atrium 16, left ventricle 18, aorta 34, inferior vena cava 42, superior vena cava 44, and right pulmonary veins 50 (including right inferior pulmonary vein 50i and right superior pulmonary vein 50s). FIGS. 16A-16B further show cardiac device 412.

An example cardiac device is referred to as cardiac device 412 in FIGS. 16A-16B. Cardiac device 412 can be any of cardiac devices 150, 200, 220, 240, 300, or 320 discussed above in reference to FIGS. 7-12.

As illustrated in FIG. 16A, cardiac device 412 can be located in an opening in common wall 64 between right inferior pulmonary vein 50i and right atrium 12, inferior vena cava 42, and/or superior vena cava 44 and implantation of cardiac device 412.

As illustrated in FIG. 16B, cardiac device 412 can be located in right inferior pulmonary vein 50i in an area where no common wall exists between right inferior pulmonary vein 50i and the right side of heart 10. As shown, cardiac device 412 can be implanted to clamp a wall of right inferior pulmonary vein 50i to a wall of right atrium 12, inferior vena cava 42, or superior vena cava 44. Cardiac device 412 can serve to hold the tissue walls together and to form a fluid-tight seal.

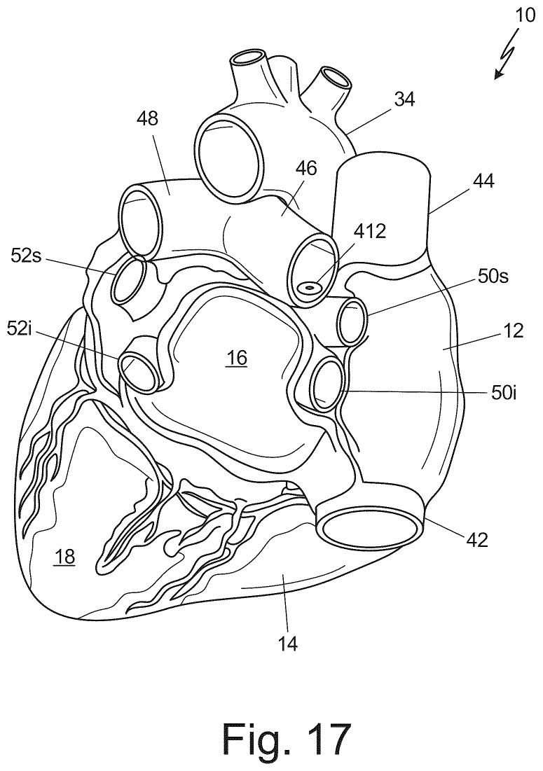

FIG. 17 is a posterior view of heart 10 having cardiac device 412 implanted in right superior pulmonary vein 50s and right pulmonary artery 46. FIG. 17 shows heart 10, right atrium 12, right ventricle 14, left atrium 16, left ventricle 18, aorta 34, inferior vena cava 42, superior vena cava 44, right pulmonary artery 46, left pulmonary artery 48, right pulmonary veins 50 (including right inferior pulmonary vein 50i and right superior pulmonary vein 50s), and left pulmonary veins 52 (including left inferior pulmonary vein 52i and left superior pulmonary vein 52s). FIG. 17 further show cardiac device 412.

An example cardiac device is referred to as cardiac device 412 in FIG. 17. Cardiac device 412 can be any of cardiac devices 150, 200, 220, 240, 300, or 320 discussed above in reference to FIGS. 7-12.

Although a common wall between right superior pulmonary vein 50s and right pulmonary artery 46 may generally not be present, examples of the present disclosure can involve forming a fluid-tight conduit between such vessels, as shown in FIG. 17. For example, the blood vessels can be clamped together using a grommet-type cardiac device as described above in FIGS. 11-12. Positioning cardiac device 412 between right superior pulmonary vein 50s and right pulmonary artery 46 permits a measurement of fluid in left atrium 16 via right superior pulmonary vein 50s and/or a measurement of fluid in right ventricle 14 via right pulmonary artery 46. The measurement of fluid in left atrium 16 and right ventricle 14 can comprise a pressure measurement. By measuring the pressure in left atrium 16 and right ventricle 14, a pressure drop across the lungs is able to be measured. Further, a blood flow can be measured at both right superior pulmonary vein 50s and right pulmonary artery 46. With a pressure drop and blood flow through the lungs, a resistance of the lungs can be calculated which can be correlated to lung function.

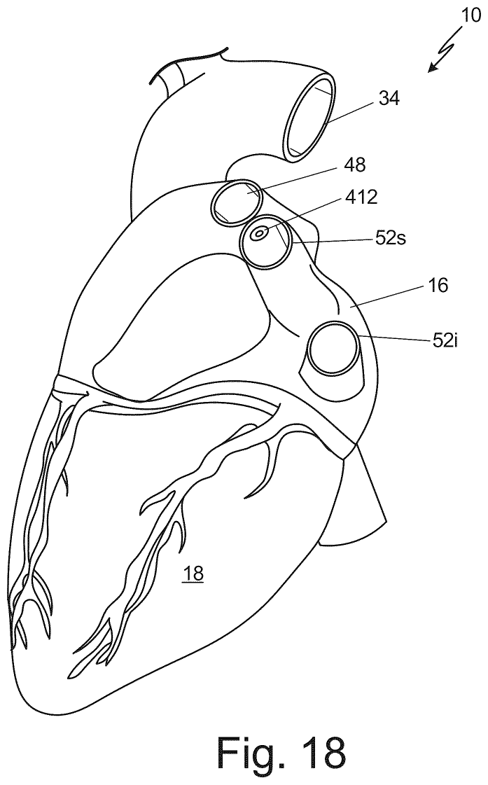

FIG. 18 is a posterior view of heart 10 having cardiac device 412 deployed between left superior pulmonary vein 52s and left pulmonary artery 48 in accordance with one or more examples. FIG. 18 shows heart 10, left atrium 16, left ventricle 18, aorta 34, left pulmonary artery 48, and left pulmonary veins 52 (including left inferior pulmonary vein 52i and left superior pulmonary vein 52s). FIG. 18 further shows cardiac device 412.

An example cardiac device is referred to as cardiac device 412 in FIG. 18. Cardiac device 412 can be any of cardiac devices 150, 200, 220, 240, 300, or 320 discussed above in reference to FIGS. 7-12.

Although a common wall between left superior pulmonary vein 52s and left pulmonary artery 48 may generally not be present, examples of the present disclosure can involve forming a fluid-tight conduit between such vessels, as shown in FIG. 18. For example, the blood vessels can be clamped together using a grommet-type cardiac device as described above in FIGS. 11-12. Positioning cardiac device 412 between left superior pulmonary vein 52s and left pulmonary artery 48 permits pressure measurement in left atrium 16 via left superior pulmonary vein 52s and right ventricle 14 via left pulmonary artery 48. As described above with respect to FIG. 17, these measurements permit a pressure drop across and blood flow through the lungs to be measured. As such, a current health of the lungs can be calculated from the pressure drop and blood flow measurements.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Detailed Embodiments

The following are non-exclusive descriptions of possible embodiments of the present invention.

A cardiac device includes a body configured to be positioned in a wall between a right pulmonary vein and a right atrium or a superior vena cava and a sensor attached to the cardiac device. The sensor includes a sensor housing, a transducer, and control circuitry in the sensor housing in electrical communication with the transducer that is configured to sense a signal from a fluid in the right pulmonary vein, the right atrium, or the superior vena cava or a signal from a heart.

The cardiac device of the preceding paragraph can optionally include, additionally and/or alternatively, any one or more of the following features, configurations and/or additional components:

Wherein the control circuitry is configured to sense a pressure of blood in the right pulmonary vein.

Wherein the pressure of blood in the right pulmonary vein is representative of a pressure of blood in a left atrium of the heart.

Wherein the control circuitry is configured to sense a pressure of blood in the right atrium.

Wherein the control circuitry is configured to sense a pressure of blood in the superior vena cava.

Wherein the pressure of blood in the superior vena cava is representative of a pressure of blood in the right atrium of the heart.

Wherein the body is configured to be positioned in a common wall between a right superior pulmonary vein and the right atrium or the superior vena cava.

Wherein the sensor is attached to a first end of the cardiac device and is configured to be positioned in the right pulmonary vein.

Wherein the sensor is attached to a second end of the cardiac device and is configured to be positioned in the right atrium or the superior vena cava.

Wherein the sensor extends through the body of the cardiac device with a first end that is configured to be positioned in the right pulmonary vein and a second end that is configured to be positioned in the right atrium or the superior vena cava.

Wherein the body of the cardiac device forms the sensor housing.

Wherein the transducer is positioned in the sensor housing.

Wherein the transducer is positioned outside of and attached to the sensor housing.

The sensor further includes an antenna positioned in the sensor housing and in electrical communication with the control circuitry, wherein the antenna is configured to communicate a signal sensed by the control circuitry to an external device.

Wherein the transducer is selected from the group consisting of a piezoelectric transducer, a strain gauge, a fiber optic pressure sensor, and combinations thereof.

Wherein the sensor is attached to the body of the cardiac device.

The cardiac device further includes a first anchor arm extending outward from a first end of the body, and a second anchor arm extending outward from a second end of the body.

Wherein the sensor is attached to the first anchor arm or the second anchor arm.

Wherein the first anchor arm is anchored to a tissue wall of the right pulmonary vein.

Wherein the second anchor arm is anchored to a tissue wall of the right atrium or the superior vena cava.

Wherein the cardiac device is a shunt device and further includes an opening extending through the body of the cardiac device that is configured to shunt blood from the right pulmonary vein to the right atrium or the superior vena cava.

Wherein the sensor is positioned in the opening and attached to the body of the cardiac device.

Wherein the cardiac device is sterilized.

A cardiac device includes a body configured to be positioned in a wall between a vein fluidly coupled to a left atrium and a right atrium or a vein fluidly coupled to the right atrium, and a sensor attached to the cardiac device. The sensor includes a sensor housing, a transducer, and control circuitry in the sensor housing in electrical communication with the transducer that is configured to sense a signal from a fluid in the vein fluidly coupled to the left atrium, the right atrium, or the vein fluidly coupled to the right atrium or a signal from a heart.

The cardiac device of the preceding paragraph can optionally include, additionally and/or alternatively, any one or more of the following features, configurations and/or additional components:

Wherein the body is configured to be positioned in a common wall between a right superior pulmonary vein and the right atrium or a superior vena cava.

Wherein the body is configured to be positioned in a wall of a right superior pulmonary vein and a wall of the right atrium or a superior vena cava.

Wherein the body is configured to be positioned in a common wall between a right inferior pulmonary vein and the right atrium, an inferior vena cava, or a superior vena cava.

Wherein the body is configured to be positioned in a wall of the right inferior pulmonary vein and a wall of the right atrium, an inferior vena cava, or a superior vena cava.

Wherein the body is configured to be positioned in a wall of a right superior pulmonary vein and a wall of a right pulmonary artery.

Wherein the body is configured to be positioned in a wall of a left superior pulmonary vein and a wall of a left pulmonary artery.

A method of determining a cardiac pressure includes inserting a catheter to a left atrium of a heart, and advancing the catheter into a right pulmonary vein to a wall between the right pulmonary vein and a superior vena cava or a right atrium. An opening is formed in the wall between the right pulmonary vein and the superior vena cava or the right atrium, and a cardiac device with a sensor is deployed into the opening. A pressure is sensed in the right pulmonary vein.

The method of the preceding paragraph can optionally include, additionally and/or alternatively, any one or more of the following features, configurations and/or additional components:

Wherein sensing the pressure in the right pulmonary vein includes sensing the pressure in the right pulmonary vein that is representative of a pressure in a left atrium.

Wherein sensing the pressure in the right pulmonary vein includes continuously sensing the pressure in the right pulmonary vein.

The method further includes sensing a pressure in the superior vena cava or the right atrium.

The method further includes anchoring the cardiac device to a wall of the right pulmonary vein and a wall of the superior vena cava or the right atrium.

The method further includes sterilizing the catheter prior to inserting the catheter to the left atrium of the heart.

The method further includes sterilizing the cardiac device prior to deploying the cardiac device.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A method of determining a cardiac pressure, the method comprising:
inserting a catheter to a left atrium of a heart;
advancing the catheter into a right pulmonary vein to a wall between the right pulmonary vein and a superior vena cava or a right atrium;
forming an opening in the wall between the right pulmonary vein and the superior vena cava or the right atrium;
deploying a cardiac device with a sensor into the opening; and
sensing a pressure of blood in the right pulmonary vein.

2. The method of claim 1, wherein sensing the pressure in the right pulmonary vein includes sensing the pressure of blood in the right pulmonary vein that is representative of a pressure of blood in a left atrium.

3. The method of claim 1, and further comprising:
sensing a pressure of blood in the superior vena cava that is representative of the pressure of blood in the right atrium or a pressure of blood in the right atrium.

4. The method of claim 1, wherein forming the opening in the wall between the right pulmonary vein and the superior vena cava or the right atrium further comprises:
forming the opening in a common wall between a right superior pulmonary vein and the right atrium or the superior vena cava.

5. The method of claim 1, wherein the sensor is a first sensor attached to a first end of the cardiac device, and further comprising:
positioning the first sensor in the right pulmonary vein.

6. The method of claim 5, wherein the cardiac device further includes a second sensor attached to a second end of the cardiac device, and further comprising:
positioning the second sensor in the right atrium or the superior vena cava.

7. The method of claim 1, wherein the sensor extends through the cardiac device, and further comprising:
positioning a first end of the sensor in the right pulmonary vein; and
positioning a second end of the sensor in the right atrium or the superior vena cava.

8. The method of claim 1,
wherein the sensor further includes:
a sensor housing; and
a transducer; and
wherein the cardiac device further includes:
control circuitry in the sensor housing in electrical communication with the transducer.

9. The method of claim 8, wherein a body of the cardiac device forms the sensor housing.

10. The method of claim 8, wherein the sensor further comprises:
an antenna positioned in the sensor housing and in electrical communication with the control circuitry, wherein the antenna is configured to communicate a signal sensed by the control circuitry to an external device.

11. A method of determining a cardiac pressure, the method comprising:
inserting a catheter to a left atrium of a heart;
advancing the catheter into a right pulmonary vein to a wall between the right pulmonary vein and a superior vena cava or a right atrium;
forming an opening in a common wall between the right pulmonary vein and the superior vena cava or the right atrium;
deploying a cardiac device with a sensor into the opening; and
sensing a pressure of blood in the right pulmonary vein that is representative of a pressure of blood in a left atrium.

12. The method of claim 11, and further comprising:
sensing a pressure of blood in the superior vena cava that is representative of the pressure of blood in the right atrium or a pressure of blood in the right atrium.

13. The method of claim 11, wherein the sensor is a first sensor attached to a first end of the cardiac device, and further comprising:
positioning the first sensor in the right pulmonary vein.

14. The method of claim 13, wherein the cardiac device further includes a second sensor attached to a second end of the cardiac device, and further comprising:
positioning the second sensor in the right atrium or the superior vena cava.

15. The method of claim 11, wherein the sensor extends through the cardiac device, and further comprising:
positioning a first end of the sensor in the right pulmonary vein; and
positioning a second end of the sensor in the right atrium or the superior vena cava.

16. The method of claim 11, and further comprising:
anchoring the cardiac device to the wall using a first anchor arm and a second anchor arm of the cardiac device, wherein the first anchor arm is anchored to a tissue wall of the right pulmonary vein, and wherein the second anchor arm is anchored to a tissue wall of the right atrium or the superior vena cava;
wherein the sensor is attached to the first anchor arm or the second anchor arm.

17. The method of claim 11, wherein the cardiac device is a shunt device, and further comprising:
positioning an opening of the shunt device in the opening between the right pulmonary vein and the superior vena cava or the right atrium; and
shunting blood from the right pulmonary vein to the superior vena cava or the right atrium;
wherein the sensor is positioned in the opening of the shunt device.

18. A method of determining a cardiac pressure, the method comprising:
inserting a catheter to a left atrium of a heart;
advancing the catheter into a right pulmonary vein to a wall between the right pulmonary vein and a right atrium or a vein fluidly coupled to the right atrium;
forming an opening in the wall between the right pulmonary vein and the right atrium or the vein fluidly coupled to the right atrium;
deploying a cardiac device with a sensor into the opening; and
sensing a pressure of blood in the right pulmonary vein that is representative of a pressure of blood in the left atrium.

19. The method of claim 18, and further comprising:
sensing a pressure of blood in the right atrium or a pressure of blood in the vein fluidly coupled to the right atrium that is representative of the pressure of blood in the right atrium.

20. The method of claim 18, wherein forming the opening in the wall between the right pulmonary vein and the right atrium or the vein fluidly coupled to the right atrium further comprises:
forming the opening in a common wall between a right superior pulmonary vein and the right atrium or a superior vena cava;
forming the opening in a wall of the right superior pulmonary vein and a wall of the right atrium or the superior vena cava;
forming the opening in a common wall between a right inferior pulmonary vein and the right atrium, an inferior vena cava, or the superior vena cava;
forming the opening in a wall of the right inferior pulmonary vein and a wall of the right atrium, the inferior vena cava, or the superior vena cava;
forming the opening in the wall of the right superior pulmonary vein and a wall of a right pulmonary artery; or
forming the opening in a wall of a left superior pulmonary vein and a wall of a left pulmonary artery.
